(19) United States
(12) Patent Application Publication
(10) Pub. No.: US 2025/0377329 A1
(43) Pub. Date: Dec. 11, 2025

(54) TMD-BIOPOLYMER COMPOSITE SENSOR FOR MANGANESE DETECTION

(71) Applicants: University of Central Florida Research Foundation, Inc., Orlando, FL (US); METAWATER Co., Ltd, Tokyo (JP)

(72) Inventors: Woo Hyoung Lee, Orlando, FL (US); Jonghyun Baik, Orlando, FL (US); Eri Hasegawa, Tokyo (JP); Naoki Kanekawa, Tokyo (JP)

(21) Appl. No.: 19/186,837

(22) Filed: Apr. 23, 2025

Related U.S. Application Data
(60) Provisional application No. 63/656,712, filed on Jun. 6, 2024.

(51) Int. Cl. G01N 27/30; C25D 9/04; G01N 27/48; G01N 33/18

(52) U.S. Cl. CPC G01N 27/308; C25D 9/04; G01N 27/307; G01N 27/48; G01N 33/18

(57) ABSTRACT

This invention relates to an electrochemical sensor for rapid, sensitive detection of manganese ions (Mn²⁺) in water. The sensor features a screen-printed carbon electrode (SPCE) substrate coated with an electrochemically deposited composite of molybdenum disulfide (MoS₂), a transition metal dichalcogenide (TMD), and chitosan, a biopolymer. The deposition process utilizes optimized potentials (+0.7 to +1.1 V) and times (30 to 900 s), ensuring uniform, stable coatings. Sensor sensitivity is maximized using a 0.2 M acetate buffer solution (pH 4.5), significantly reducing interference from competing ions and dissolved organic carbon. Detection employs square wave adsorptive cathodic stripping voltammetry (SWAdCSV), reliably quantifying trace Mn²⁺ concentrations below regulatory limits. Minimal interference from common ions except ferrous iron (Fe²⁺) and simplified calibration procedures for various water sources enable on-site, real-time Mn²⁺ monitoring. The invention offers a cost-effective, practical solution for environmental and potable water testing.

### PRIORITY CLAIM

This application claims priority to U.S. Provisional Patent Application 63/656,712 filed Jun. 6, 2024 and entitled “TMD-Biopolymer Composite Sensor for Manganese Detection.”

### 1. FIELD OF THE INVENTION

This invention relates to the field of electrochemical sensors, and more particularly, to the design and fabrication of low-cost, high-sensitivity sensors for the selective electrochemical detection and quantification of manganese ions (Mn²⁺) in water, utilizing screen-printed carbon electrodes (SPCE) modified with composites comprising transition metal dichalcogenides (TMDs), specifically molybdenum disulfide (MoS₂), integrated with biopolymer matrices, notably chitosan.

### 2. BRIEF DESCRIPTION OF THE PRIOR ART

Manganese ions (Mn²⁺) are vital micronutrients required for essential metabolic functions, yet excessive exposure can have significant adverse health implications, including neurodegenerative disorders in adults, such as manganism, and severe neurodevelopmental impairments in children. Recognizing these risks, authoritative bodies such as the World Health Organization (WHO) recommend a stringent threshold for manganese in drinking water, setting provisional guidance at approximately 0.08 mg/L, particularly emphasizing protection for infants, who are the most vulnerable demographic group. Concurrently, the United States Environmental Protection Agency (U.S. EPA) maintains a secondary maximum contaminant level (MCL) guideline at 0.05 mg/L. Consequently, accurate, frequent, and point-of-use monitoring of manganese ion concentrations in water sources is critical for safeguarding public health and adhering to regulatory compliance, making it necessary to develop rapid and portable detection systems.

Traditional analytical methods for detecting manganese ions in water primarily employ inductively coupled plasma mass spectrometry (ICP-MS), atomic absorption spectroscopy (AAS), or related spectroscopic techniques, known for their high accuracy, sensitivity, and low detection limits (around 0.1 μg/L). Despite these advantages, these approaches are inherently limited by several practical drawbacks. They require large, expensive instrumentation and extensive operational training, which restricts their deployment to centralized laboratory environments. Furthermore, the extended turnaround time for analysis, often weeks to months due to batching and transportation, makes timely, on-site monitoring infeasible, severely limiting their utility in emergency response scenarios and routine point-of-use measurements.

Given the inherent limitations of conventional spectroscopic analytical techniques, electrochemical sensing methods have emerged as attractive alternatives for manganese ion detection due to their ability to offer compact, cost-effective, portable, and highly sensitive solutions suitable for real-time, on-site monitoring. Specifically, the current invention addresses the prior art limitations by developing an innovative electrochemical sensor composite using a novel fabrication process comprising a transition metal dichalcogenide (TMD)—particularly molybdenum disulfide (MoS₂)—and chitosan biopolymer deposited electrochemically onto a screen-printed carbon electrode (SPCE). Notably, this invention utilizes cathodic stripping voltammetry (CSV), particularly square wave adsorptive cathodic stripping voltammetry (SWAdCSV), for enhanced sensitivity and specificity in detecting trace levels of Mn²⁺ in diverse water sources, thereby providing significant improvement in both portability and ease-of-use compared to existing laboratory-based methodologies.

Existing electrochemical sensor technologies in the prior art typically rely on electrode modifications with materials such as graphene oxide (GO), reduced graphene oxide (rGO), carbon nanotubes, or mercury-based electrodes. For instance, prior work disclosed a sensor utilizing an rGO/MoS₂/chitosan composite on a glassy carbon electrode (GCE) specifically for detecting lead (Pb²⁺) ions, highlighting the improved conductivity from rGO and the enrichment capabilities of chitosan. While effective for lead, the reliance on graphene materials may introduce instability, aggregation concerns, and complexity in sensor preparation. Furthermore, other prior art sensors for manganese detection often rely on precious metal electrodes, such as platinum or palladium, limiting their affordability and scalability for broad environmental deployment. In stark contrast, the present invention intentionally excludes graphene and carbon nanotubes, focusing exclusively on an electrodeposited composite consisting essentially of MoS₂ and chitosan deposited directly onto a low-cost, disposable SPCE substrate, thus providing a simplified, reproducible, and economical sensing device specifically optimized for manganese ion detection.

The invention further distinguishes itself from prior electrochemical sensing techniques by explicitly employing exfoliated MoS₂ nanosheets combined with chitosan derived from partial deacetylation of chitin. MoS₂ nanosheets provide an increased catalytic surface area, enhanced electrical conductivity, and efficient catalytic activity due to their unique layered structure, comprising covalent Mo—S bonds and weak van der Waals forces between layers. Moreover, the method of electrochemical deposition utilized in this invention, characterized by optimized deposition potentials (+0.7 V to +1.1 V) and times (30 to 900 seconds), produces uniform, stable, and reproducible coatings. The invention explicitly addresses prior art limitations by avoiding complex physical deposition techniques, photolithography, or toxic heavy metals such as mercury, employing instead a biocompatible, sustainable chitosan-MoS₂ composite to achieve superior sensitivity, stability, and operational simplicity in manganese ion detection.

Regarding the detection method, anodic stripping voltammetry (A SV), common in prior art sensors for heavy metals, is challenging for manganese ions due to the very negative potentials required, causing hydrogen interference and signal instability. Therefore, the present invention strategically utilizes cathodic stripping voltammetry (CSV), particularly square wave adsorptive cathodic stripping voltammetry (SWAdCSV), uniquely optimized to detect manganese ions without the interference associated with traditional ASV methods. The electrochemical method described here significantly improves selectivity, avoids interferences from competing ions (e.g., Cu²⁺, Pb²⁺, and Zn²⁺), and is optimized specifically to measure free-state manganese ions. This approach enhances detection reliability and accuracy, directly addressing critical limitations cited in prior art methods while providing rapid, portable, and field-deployable manganese ion measurements, suitable for environmental monitoring and compliance with public health standards.

### SUMMARY OF THE INVENTION

The long-standing but heretofore unfulfilled need for a rapid, sensitive, low-cost electrochemical sensor for manganese ion (Mn²⁺) detection in water is now met by the novel invention disclosed herein. The invention specifically provides an electrochemical sensor composite consisting essentially of molybdenum disulfide (MoS₂) and chitosan deposited directly onto a screen-printed carbon electrode (SPCE). MoS₂ is preferably present as exfoliated nanosheets. The chitosan employed herein is derived specifically from the partial deacetylation of chitin.

In an embodiment, MoS₂ and chitosan are combined in a composite solution prior to deposition, with the solution comprising MoS₂ dissolved in isopropyl alcohol and chitosan dissolved in an acetate buffer maintaining a stable pH of approximately 4.5. This composite solution explicitly excludes graphene and carbon nanotubes to ensure simplicity, stability, and reduced manufacturing complexity. The composite is electrochemically deposited directly onto the SPCE substrate within a precisely controlled deposition potential range of about +0.7 V to +1.1 V for durations between 30 and 900 seconds. The optimized electrodeposition parameters yield a uniform, stable composite coating, with the resulting deposition surface area on the SPCE specifically about 7 mm².

The invention further provides an electrochemical sensing apparatus configured specifically for detecting manganese ions. The apparatus comprises: (a) the SPCE substrate; (b) a working electrode coated with the aforementioned composite of MoS₂ and chitosan; (c) a reference electrode configured to maintain a stable potential relative to the working electrode, preferably an Ag/AgCl electrode; (d) a counter electrode configured to complete the electrical circuit; and (e) an electrochemical analyzer device specifically configured to perform square wave adsorptive cathodic stripping voltammetry (SWAdCSV). The electrochemical analyzer operates within optimized voltammetric parameters, specifically an amplitude of between about 5 to 150 mV and a frequency range of between about 5 to 200 Hz, thereby maximizing sensitivity and reliability.

Advantageously, the apparatus is specifically designed for portable, field-deployable use and is integrated onto a disposable sensor strip fabricated from low-cost plastic substrates. Notably, the composite sensor system requires no additional chemical ligand or reagent for manganese ion accumulation, simplifying its practical utility and reducing costs associated with on-site analysis.

The invention also discloses a method for detecting manganese ions in water. This method comprises: (a) preparing a composite solution by dissolving MoS₂ nanosheets in isopropyl alcohol and chitosan in an acetate buffer; (b) electrochemically depositing the composite solution directly onto the SPCE substrate, particularly at an optimized deposition potential of about +0.7 V to +1.1 V (preferably around +0.9 V) for between about 30 to 900 seconds; (c) immersing the deposited electrode into a water sample whose pH has preferably been adjusted to approximately 4.5 using acetate buffer; (d) applying square wave adsorptive cathodic stripping voltammetry (SWAdCSV) specifically using optimized parameters, an amplitude of between about 5 to 150 mV, and frequency between about 5 to 200 Hz; and (e) detecting and quantifying manganese ions at trace concentrations, achieving detection sensitivity with a limit of detection (LOD) below approximately 1 μg/L Mn²⁺.

Preferably, the electrode prepared by this method has an electrode surface area specifically around 7 mm², and the electrode itself may be reused following a regeneration step involving the application of a cleaning potential to remove residual deposits, thereby enhancing cost-effectiveness and environmental sustainability. Furthermore, the deposited composite explicitly excludes graphene and carbon nanotubes, simplifying manufacturing and improving stability.

The invention additionally encompasses a method of fabricating the electrochemical sensor composite for manganese ion detection. The fabrication method comprises electrochemically depositing the composite consisting essentially of MoS₂ and chitosan onto the SPCE substrate. The fabrication method preferably involves mixing MoS₂ and chitosan solutions in equal volumes before deposition. Electrochemical deposition of the composite occurs specifically within a potential range of about +0.7 to +1.1 V for between about 30 to 900 seconds. Additionally, the fabrication method preferably includes drying each deposited composite layer for approximately 4 minutes under heat to ensure stable film formation. MoS₂ is preferably employed as exfoliated nanosheets, and the chitosan is explicitly prepared from chitin by partial deacetylation. Further, the resulting sensor is fabricated onto disposable SPCE substrates constructed from inexpensive plastic materials, facilitating broad, low-cost implementation.

The invention uniquely combines these specific materials and optimized electrochemical processes, offering a significant advancement over existing analytical methods. Unlike conventional laboratory-based methods, the described electrochemical sensor and methods herein provide a compact, efficient, and real-time monitoring capability, greatly enhancing the practicality and accessibility of manganese ion testing across diverse environmental and drinking water applications.

Additional aspects and advantages of the present disclosure will become readily apparent to those skilled in the art from the following detailed description, wherein only illustrative embodiments of the present disclosure are shown and described. As will be appreciated, the present disclosure is capable of other and different embodiments, and its several details are subject to modifications in various obvious respects without departing from the disclosure. Accordingly, the drawings and descriptions herein are to be regarded as illustrative in nature, and not restrictive.

The invention accordingly comprises the features of construction, combination of elements, and arrangement of parts exemplified herein, with the scope of the invention clearly indicated in the appended claims.

### BRIEF DESCRIPTION OF THE DRAWINGS

For a fuller understanding of the invention, reference should be made to the following detailed description, taken in connection with the accompanying drawings, in which:

FIG. 1 schematic diagram of the Mn²⁺ determination using cathodic stripping voltammetry (CSV).

FIGS. 2(a-c) show square wave adsorptive cathodic stripping voltammetry technique graphs of (a) bare SPCE, (b) chitosan-coated SPCE sensor, and (c) TMD-biopolymer-modified SPCE sensor with different Mn²⁺ concentrations (10-2,000 ppb) in 0.1 M acetate buffer solution at pH 4.5. Deposition potential: +0.7 V, Deposition time: 300 s, Amplitude: 25 mV, and Frequency: 25 Hz.

FIG. 3 is a graph view showing representative calibration curves of the three electrodes.

FIGS. 4(a-e) show XPS analysis of TMD-biopolymer-modified SPCE sensor. (a) M o 3d spectra, (b) Mn2p spectra, (c) C 1s spectra, (d) O 1s spectra, and (e) N 1s spectra.

FIGS. 5(a-b) show SEM images of (a) bare SPCE and (b) TMD-biopolymer-modified SPCE sensor (at 1.00 K magnification).

FIGS. 6(a-d) show bar charts of optimization of experimental settings of the SWAdCSV. Mn²⁺ peaks in various (a) deposition potentials, (b) deposition times, (c) amplitudes, and (d) frequencies. (50 μg L⁻¹ of Mn²⁺, 0.1 M acetate buffer solution (pH 4.5)).

FIGS. 7(a-b) are images of working electrodes (a) before and (b) after chitosan coating on SPCE. The surface area of the chitosan coating was 7 mm².

FIG. 8 is an image of an experimental setup of the electrochemical sensor evaluation.

FIG. 9 is a graph showing a corresponding curve of the MoS₂-chitosan-modified electrode for Mn²⁺ detection in the lake water sample.

FIG. 10 is a graph showing sensor sensitivity comparison at varying acetate buffer concentrations (0.1 M versus 0.2 M).

FIG. 11 is a bar chart showing the effect of potential interfering ions (Cu²⁺, Pb²⁺, Mg²⁺, Ca²⁺, Zn²⁺, and Fe²⁺) on the manganese detection signal using the TMD-biopolymer-coated SPCE sensor.

FIG. 12 is a bar chart illustrating the influence of bicarbonate alkalinity on Mn²⁺ detection peak currents.

FIG. 13 is a graph showing simplified calibration curves for Mn²⁺ detection using diluted real-water samples.

FIG. 14a is a graph showing square wave adsorptive cathodic stripping voltammetry (SWAdCSV) results for 50 μg L⁻¹ Mn²⁺ spiked Lake C water samples using varying acetate buffer (AcB) dilution ratios (0-100%) to assess signal response across buffer strengths.

FIG. 14b is a graph showing the calibration curve for Mn²⁺ detection in Lake C water using 5% 0.2 M AcB dilution, validating sensitivity of the TMD-biopolymer-coated SPCE sensor in lightly buffered natural water.

FIG. 15a is a graph showing simplified calibration curves for low-range Mn²⁺ concentrations (0-20 μg L⁻¹) using three real water sources (tap water, Lake C, and Lake E), all diluted with 95% of 0.2 M AcB.

FIG. 15b is a graph showing simplified calibration curves for high-range Mn²⁺ concentrations (20-100 μg L⁻¹) under the same conditions and sources, confirming consistent sensor response across diverse water matrices.

FIG. 16a is a graph showing site-specific calibration curves for each real water sample (tap, Lake C, and Lake E) in the low Mn²⁺ concentration range (0-20 μg L⁻¹), illustrating unique matrix effects.

FIG. 16b is a graph showing site-specific calibration curves for each real water sample in the high Mn²⁺ concentration range (20-100 μg L⁻¹), further validating the need for and performance of individualized calibration in varied water chemistries.

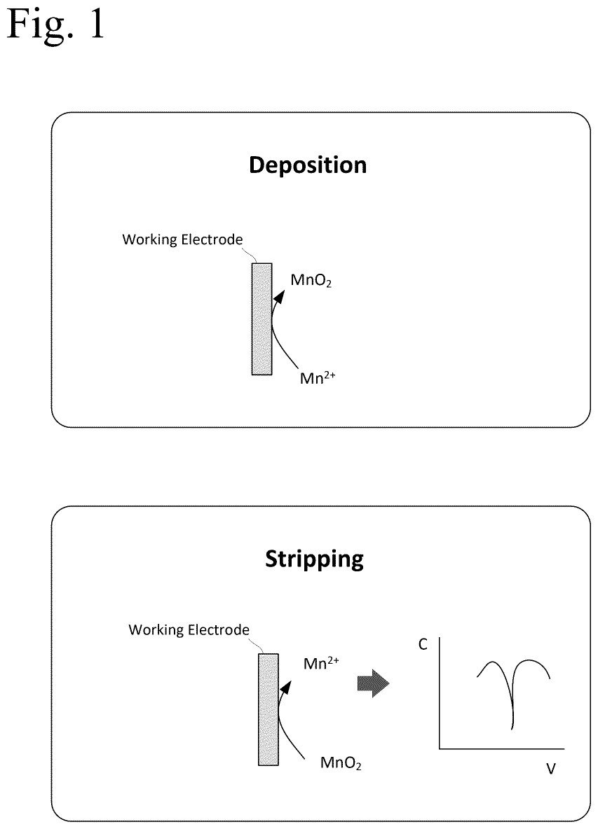

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that one skilled in the art will recognize that other embodiments may be utilized, and it will be apparent to one skilled in the art that structural changes may be made without departing from the scope of the invention.

As such, elements/components shown in diagrams are illustrative of exemplary embodiments of the disclosure and are meant to avoid obscuring the disclosure. Any headings, used herein, are for organizational purposes only and shall not be used to limit the scope of the description or the claims.

Furthermore, the use of certain terms in various places in the specification, described herein, is for illustration and should not be construed as limiting. For example, any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Therefore, a reference to first and/or second elements does not mean that only two elements may be employed there or that the first element must precede the second element in some manner. Also, unless stated otherwise a set of elements may comprise one or more elements

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” “in embodiments,” “in alternative embodiments,” “in an alternative embodiment,” or “in some embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items.

Referring in general to the following description and accompanying drawings, various embodiments of the present disclosure are illustrated to show its structure and method of operation. Common elements of the illustrated embodiments may be designated with similar reference numerals.

For the detection of Mn²⁺, an electrochemical system that comprises a three-electrode design was used, the working electrode (WE), the counter electrode (CE), and the reference electrode (RE). The electrode for the modification in this study was the WE. MoS₂ and chitosan were electrochemically deposited on a screen-printed carbon electrode (SPCE) (RRPE1001C, Pine Research Instrumentation, Durham, NC, USA) as a WE. The MoS₂ solution contained 2 mg of MoS₂ powder (98%, 1317335, Sigma-Aldrich, St. Louis, MO, USA) in 50 mL isopropyl alcohol (IPA) (99%, 67-63-0, Hexeal, Norwich, UK) followed by the previous work. The biopolymer solution was prepared by dissolving 24 mg of chitosan (18-601-571, Thermo Scientific, Waltham, MA, USA) in 20 mL of 0.1 M acetate buffer solution. The MoS₂-chitosan solution was prepared by mixing these two solutions in a 1:1 ratio. Electrodeposition of the appropriate solutions was conducted using a DC supply (PalmSens4, PalmSens, Houten, Netherlands) at −100 mA/cm² for 60 s of the deposition time. Carbon WE and CE were used which were included at the SPCE. An Ag/AgCl electrode (NC9753402, CH Instruments, Austin, TX, USA) was used as the RE

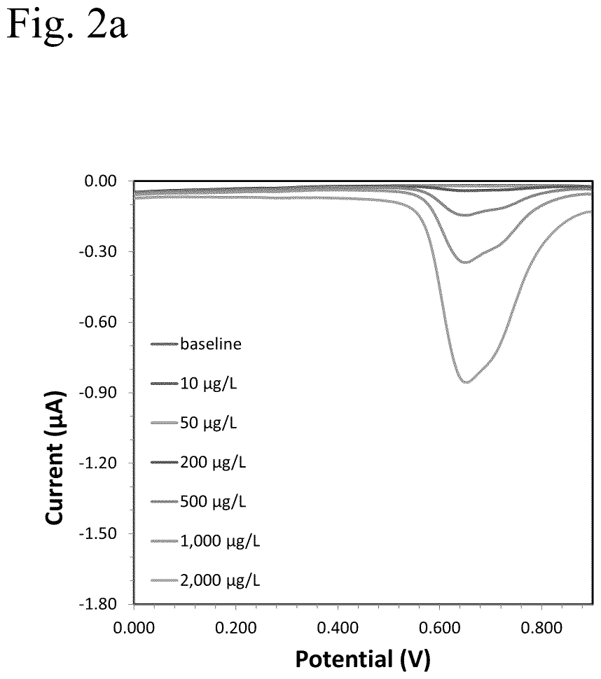

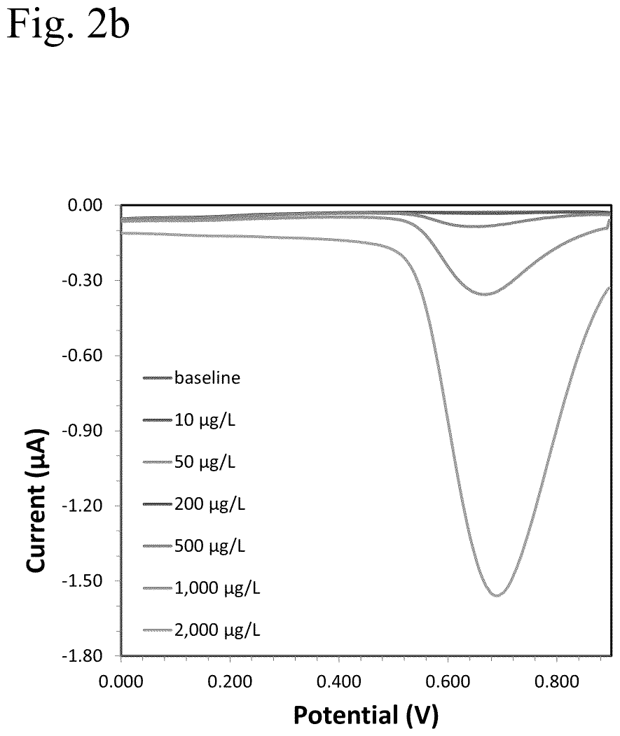

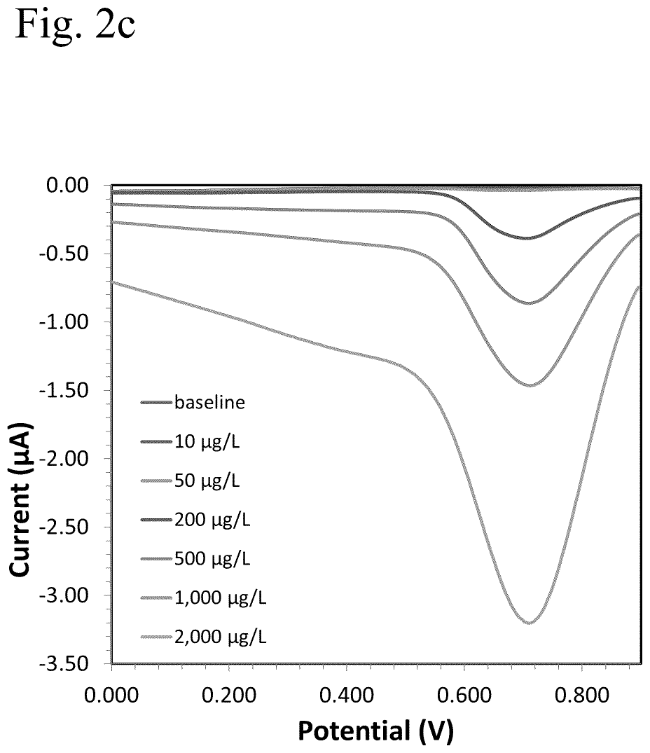

The TMD-biopolymer-modified SPCE sensor was developed by the electrodeposition of MoS₂ and chitosan, and its detection of Mn²⁺ by square wave adsorptive cathodic stripping voltammetry technique (SWAdCSV) was compared to a bare SPCE and a chitosan-only coated SPCE sensor. 50 mL of 0.1 M acetate buffer (pH 4.5) was used as the media. The initial operational parameters were ±0.7 V of deposition potential, 300 s of deposition time, 25 mV of amplitude, and 25 Hz of frequency, respectively. FIG. 2(a-c) shows the SWA dCSV results of three electrodes for Mn²⁺ detection with various Mn²⁺ concentrations (0 to 2,000 μg L⁻¹). The height of the peak has been increased when chitosan and MoS₂ were coated compared to the bare SPCE. For example, at the highest concentration of Mn²⁺ in this study (2,000 μg L⁻¹), the peak of the SWA dCSV by bare SPCE, chitosan-coated SPCE, and TMD-biopolymer-modified SPCE were 749±49.50, 1162±7.98, 2318±103.24 nA, respectively.

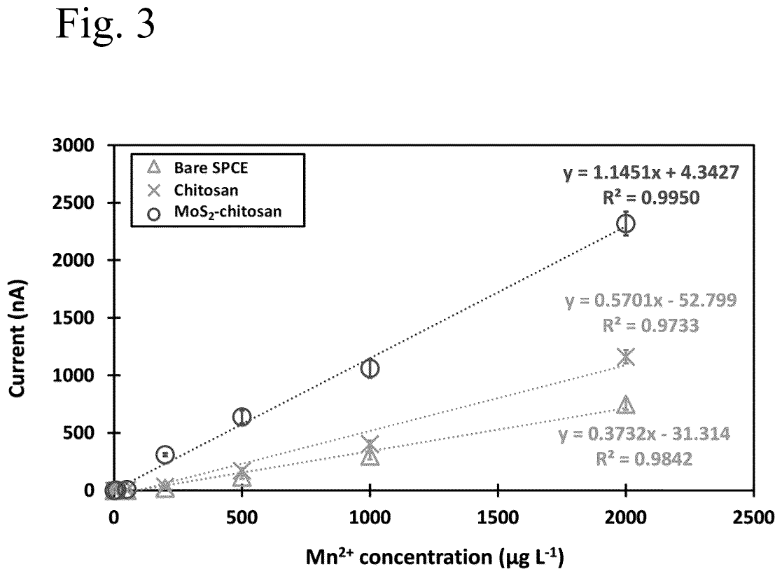

FIG. 3 shows the representative calibration curves of the three electrodes. Bare SPCE was evaluated for the Mn²⁺ detection for the control of this study. The corresponding calibration plot and correlation coefficient for the bare SPCE for the Mn²⁺ detection was Ip=0.3732x−31.314 (R²=0.9842). The calibration curve of the chitosan-coated SPCE sensor was Ip=0.5701x−52.799 (R²=0.9733), indicating 1.58 times higher sensitivity toward Mn²⁺ than the control (bare SPCE). The increase in sensitivity by the coating of chitosan suggests that chitosan as a biopolymer can help the adsorption of metal ions (Mn²⁺) due to the presence of the amino group (—NH₂). Furthermore, the presence of hydroxide group (—OH) makes the working electrode more hydrophilic which would further enhance the sensitivity of the sensor. Lastly, the TMD-biopolymer-modified SPCE sensor was evaluated and compared to both bare SPCE and chitosan-coated SPCE sensors. The sensitivity of the sensor was greatly improved 3 times higher than the control electrode and 2 times higher than the chitosan-coated SCPE sensor, respectively. The corresponding calibration plot and correlation coefficient was Ip=1.1451x+4.3427 (R²=0.9950) for the SPCE sensor. The LODs were calculated as 2.53, 1.15, and 0.98 μg L⁻¹ for bare SPCE, chitosan-coated SPCE sensor, and TMD-biopolymer-modified SPCE sensor, respectively. This result shows that the coating of the TMD and biopolymer greatly improved the stability of the electrodes during Mn²⁺ detection due to its high binding affinity between divalent ions and their sulfur sites.

### Real Water Sample Applications and Validations

The sensor's capability was further validated using real-world samples from two lakes and municipal tap water. Comparative analyses with ICP-MS revealed that the TMD-biopolymer-coated SPCE sensor accurately detected spiked Mn²⁺ concentrations, achieving recoveries of 94.76-97.25% for tap water, 96.00-94.02% for Lake E, and 97.21-107.16% for Lake C samples. These recoveries demonstrate the sensor's robustness and reliability across diverse environmental water sources. Consequently, the invention presents an effective, practical alternative to traditional laboratory-based Mn²⁺ monitoring methods, suitable for field-deployable, rapid detection scenarios.

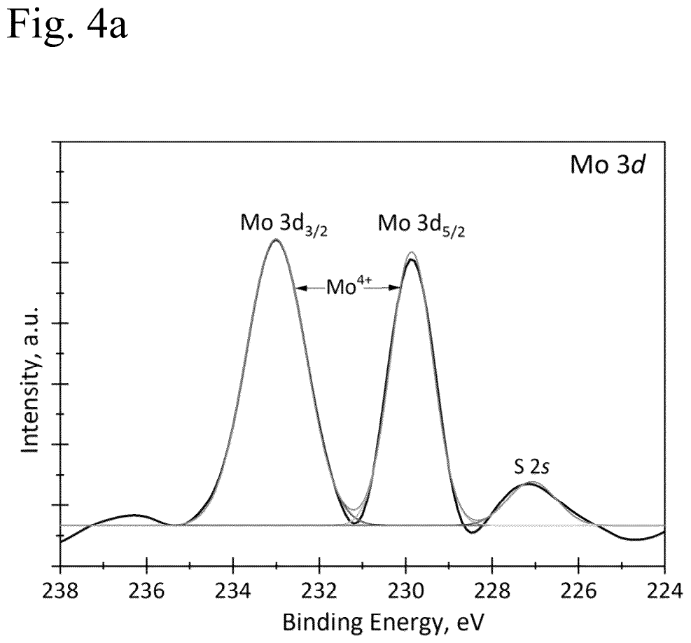

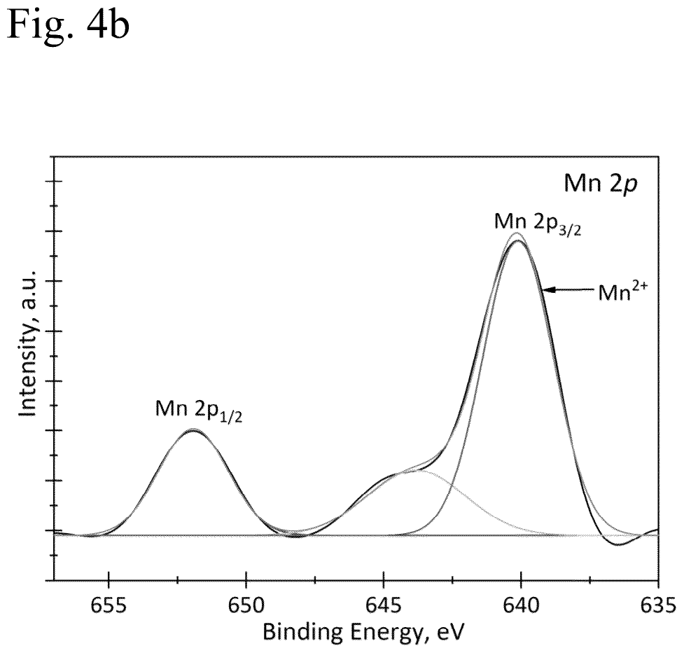

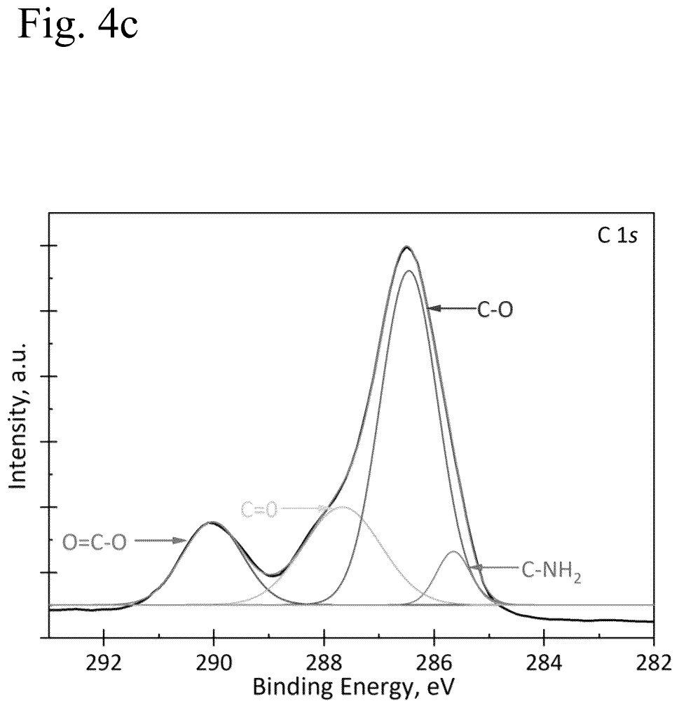

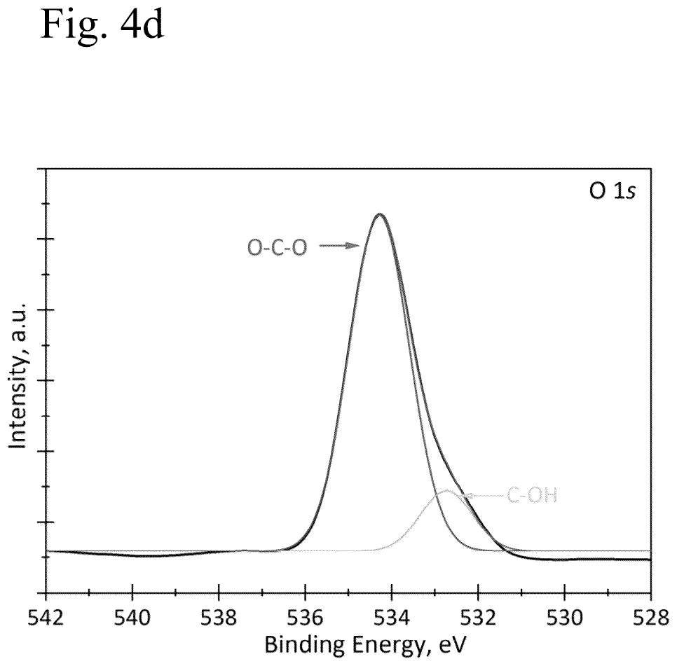

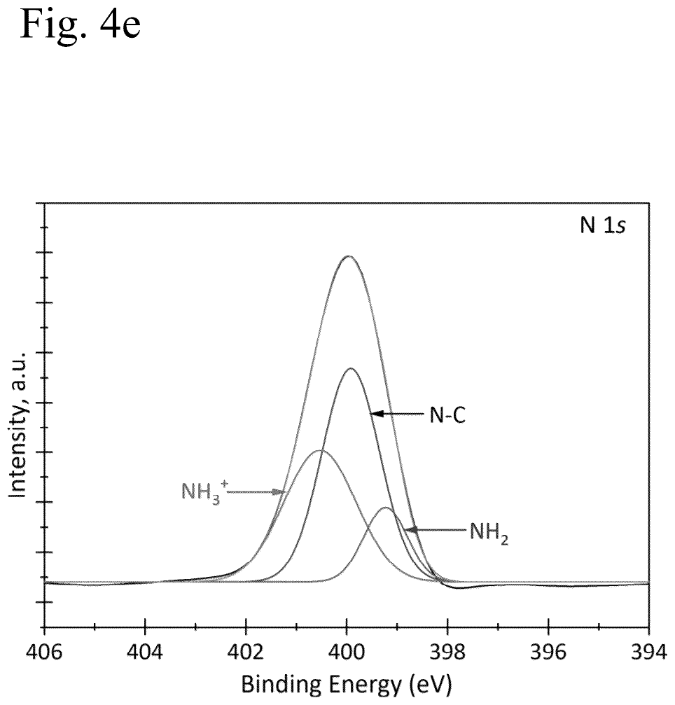

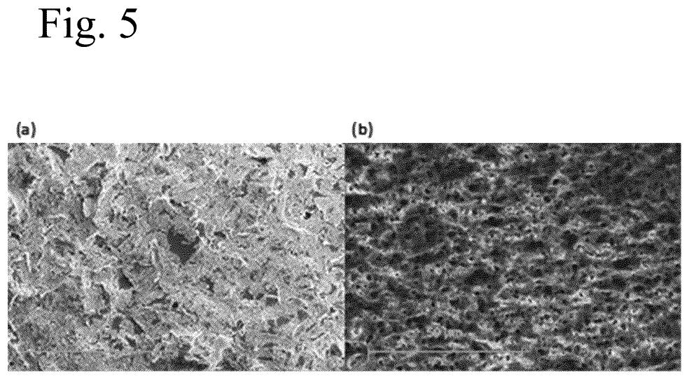

The TMD-biopolymer-modified SPCE sensor was characterized using X-ray photoelectron spectroscopy (XPS) and scanning electron microscopy (SEM) analysis. XPS analysis was used to examine the MoS₂-chitosan-coated SPCE sensor (after the detection of Mn²⁺) (FIG. 4). The analysis detected the presence of carbon (C 1s), oxygen (O 1s), nitrogen (N 1s), manganese (Mn²p), molybdenum (M o 3d), and sulfur (S 2s) was identified. In FIG. 4(a), a detailed M o 3d spectrum is depicted, highlighting the presence of two distinguishable peaks. The splitting of Mo into Mo 3d5/2 and Mo 3d3/2 spin orbitals was observed at binding energies of 229.89 eV and 232.99 eV, respectively. The width of peak separation signifies the energy associated with exchange interactions, which can be attributed to the material's oxidation state. The energy gap of 3.1 between the split orbitals suggests an oxidation state of +4, confirming the presence of MoS₂. Additionally, the scan identified a nearby S 2s peak at 227.19 eV, providing further evidence for the existence of MoS₂. The measured binding energies are in agreement with reported values elsewhere. FIG. 4(b) shows the Mn²p high-resolution spectrum of the sample. Two distinct spin orbital splitting of Mn²p3/2 and Mn²p1/2 at 640.15 eV and 651.77 eV respectively with an energy difference of 11.62 eV, indicating an oxidation state of +2. Additionally, the presence of satellite features is only observed in M nO as reported in the literature. FIG. 4(c) shows C 1s spectra divided into individual peaks at 285.4, 286.5, 287.7, and 290.1 which are assigned to C—NH₂, C—O, C═O, and O═C—O respectively. O 1s high-resolution spectra in FIG. 4(d). Two peaks at 532.6 eV and 534.1 eV indicate the presence of the C—OH and O—C—O group arising from the chitosan compound. The N 1s high-resolution spectra show a prominent peak of N—C at 399.9 eV and two secondary peaks of NH 3+ and NH₂ at 400.5 eV and 399.03 eV were reported, arising from the Chitosan compound.

Further optimization was conducted to evaluate the influence of acetate buffer (AcB) concentration on Mn²⁺ detection sensitivity. Comparisons between 0.1 M and 0.2 M A cB solutions (both at pH 4.5) revealed that the 0.2 M condition yielded approximately 1.31-fold greater sensitivity, with corresponding calibration equations Ip=4.0595x+92.374 (R²=0.9852) and Ip=5.3051x+217.51 (R²=0.9615), respectively.

Additionally, the impact of potential ion interferences was assessed using Cu²⁺, Pb²⁺, Mg²⁺, Ca²⁺, Zn²⁺, and Fe²⁺ at 50 μg/L. All but Fe²⁺ showed minimal effect (<20% deviation in peak current); Fe²⁺ notably reduced the Mn²⁺ signal by ˜69.58%, likely due to rapid chemical reduction of MnO₂ to Mn²⁺.

The sensor's performance under varying alkalinity was also evaluated. Increasing bicarbonate levels from 0 to 200 mg/L as CaCO₃ resulted in a notable drop in Mn²⁺ peak currents, with levels above 120 mg/L reducing sensitivity significantly. These findings confirm that for high-alkalinity waters, dilution with 0.2 M AcB improves sensor performance.

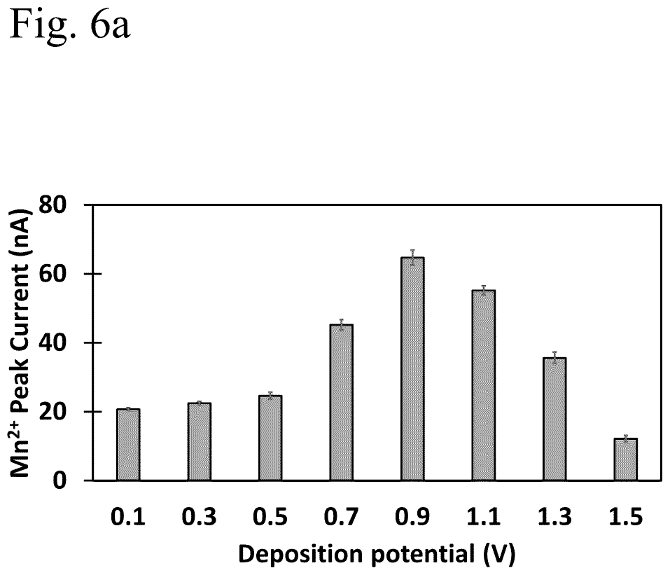

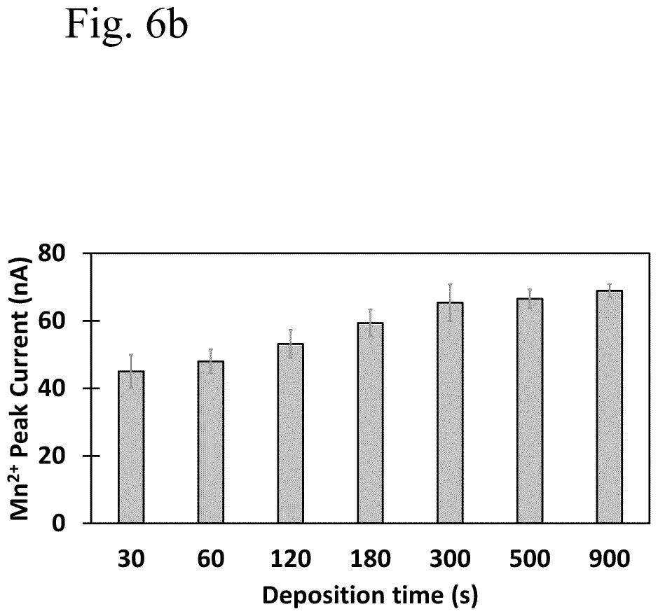

To optimize the SWAdSCV for the electrochemical Mn²⁺ detection, the effect of the operational parameters for SWA dCSV on Mn²⁺ peak currents (nA) was evaluated using the TMD-biopolymer-coated SPCE sensor (FIG. 6). This includes deposition potential (V), deposition time(s), amplitude (mV), and frequency (Hz). A fixed Mn²⁺ concentration (50 μg L⁻¹) was used, and the test solution was 0.1 M acetate buffer at pH 4.5.

For the effect of the deposition potential, eight different deposition potentials (+0.1, +0.3, +0.5, +0.7, +0.9, +1.1, +1.3, and +1.5 V) were compared for detecting 50 μg L⁻¹ Mn²⁺ in the test solution. The other parameters were set as previously (i.e., 300 s of deposition time, 25 mV of amplitude, and 25 Hz of frequency). Increasing the deposition potential up to +0.9 V increased current peaks which was up to 65 nA (FIG. 6(a)). However, the current peaks have decreased after +1.1 V of the deposition potential. It is reported that high positive deposition potentials result in the oxidation of H 20 which interferes with the MnO₂ deposition. Thus, +0.9 V of the deposition potential was selected as an optimal potential for the Mn²⁺ deposition step.

Seven different deposition times (30, 60, 120, 180, 300, 500, and 900 s) were also compared using this optimal potential. It was expected that the longer the deposition time, the more sensitive Mn detection by SWAdCSV due to the amplified amount of Mn²⁺ deposited on the WE over time. With increased deposition times from 30 and 300 s, the peak currents toward 50 μg L⁻¹ Mn²⁺ also increased from 44 to 68 nA. However, after a deposition time of 300 s, the peak currents did not increase significantly, even with three times the deposition time (i.e., 900 s). Therefore, 300 s was selected for the optimal deposition time.

Under the selected deposition potential and time (i.e., +0.9 V and 300 s), seven different amplitudes (5, 10, 25, 50, 75, 100, and 150 mV) were evaluated. The maximum peak current of 192 nA was obtained at 100 mV of amplitude and a slightly lower peak current of 181 nA was found at 75 mV of amplitude. Since the peak showed an ambient noise because of the amplitude property at 100 mV of amplitude, 75 mV amplitude was selected.

Lastly, after fixing the three parameters (i.e., +0.9 V, 300 s, and 75 mV), nine different frequencies (5, 10, 25, 50, 75, 100, 125, 150, and 200 Hz) were compared to investigate the effect of frequency on electrochemical Mn²⁺ detection. The current peak significantly increased with the increase in frequency. However, increasing it to more than 50 Hz resulted in ambient noise, resulting in 50 Hz as the optimal parameter for frequency. Overall, the optimized operating parameters for SWA dCSV using the TMD-biopolymer-coated SPCE sensor are +0.9 V of deposition potential, 300 s of deposition time, 75 mV of amplitude, and 50 Hz of frequency.

The developed TMD-biopolymer-coated SPCE sensor's performance was compared with other reported electrochemical sensors for Mn²⁺ detection, as shown in Table 1. The developed SPCE sensor provided a comparable sensitivity using low-cost materials such as MoS₂ and chitosan.

TABLE 1

| Electrochemical sensor | | | LOD | |
|---|---|---|---|---|
| WE | CE | RE | (μg L⁻¹) | Media (pH) |
| MoS₂-chitosan | Carbon | Ag/AgCl | 0.77 | 0.2M acetate buffer (4.5) |
| Indium tin | Pt | Ag/AgCl | 0.06 | 0.2M acetate buffer (5.0) |
| oxide-SSEBS | | | | |
| Pt | Pt | Pt | 0.56 | Tap water (4.7) |
| Pt | Pt | Ag/AgCl | 0.90 | 0.2M acetate buffer (5.5) |
| Pd | Pd | Cu/CuCl₂ | 18.30 | 0.1M borate buffer (9.0) |

The conventional analytical methods used to measure Mn²⁺ in water samples were based on either spectrometry (e.g., inductively coupled plasma mass spectrometry [ICP-M S]) or spectroscopy (e.g., atomic absorption spectroscopy [AAS]). They are characterized by high accuracy, sensitivity, and a low limit of quantification (LOQ) of 0.1 μg L⁻¹. Despite the strengths, these methods require bulky and expensive instruments as well as highly trained personnel, indicating only centralized laboratories are capable of housing and maintaining these instruments. Moreover, the turn-around time for analysis can extend to months or longer if samples are batched to save on shipping and analysis costs. The invention can decrease the fabrication/production cost per one sensor with improved sensitivity due to a synergetic effect of biopolymer on heavy metal holding. One unit electrode price is expected less than $10.

Among currently available analytical methods, environmental monitoring systems based on electrochemistry are considered complementary to the traditional techniques (e.g., Inductively coupled plasma (ICP) and Atomic absorption spectroscopy (AAS)), promising inexpensive and portable instruments. Particularly, square-wave anodic stripping voltammetry (SWA SV) based on metallic mercury (Hg) has been well-defined for heavy metal detection. However, due to the toxicity of Hg, environmentally benign materials that can replace Hg for heavy metal sensing materials are highly sought after. One of the most well-studied electrode materials is bismuth (Bi) due to its ability to form alloys with various heavy metals, wide potential window applicable to electrochemical detection, and low toxicity. Despite the excellent compatibility of Bi, when fabricating microsensors using Bi, the brittleness and detachment still impose practical problems for industrial applications, which have been well addressed in this invention. In this invention, TDM/biopolymer is used as a sensing material and the square wave adsorptive cathodic stripping voltammetry technique (SWAdCSV) was utilized as a reliable electrochemical analytical technique for accurate measurements of Mn²⁺ with the presence of other ions in various water systems (e.g., drinking water, lake water, and wastewater).

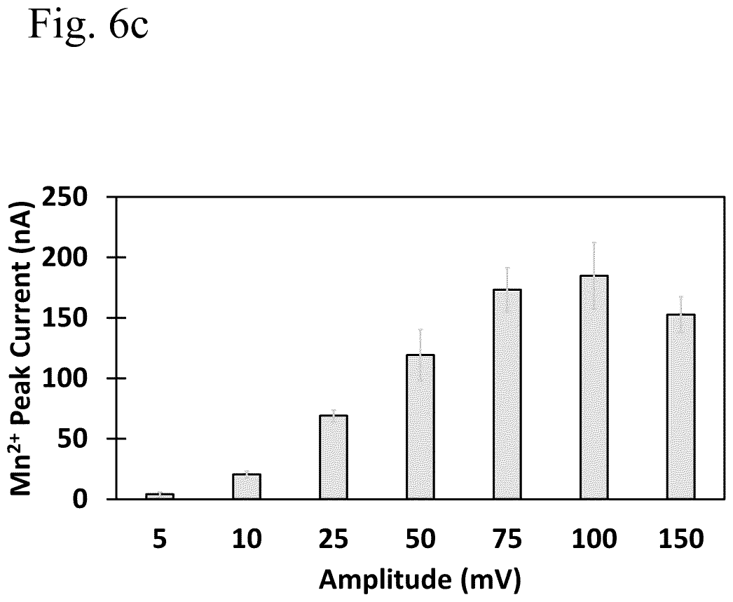

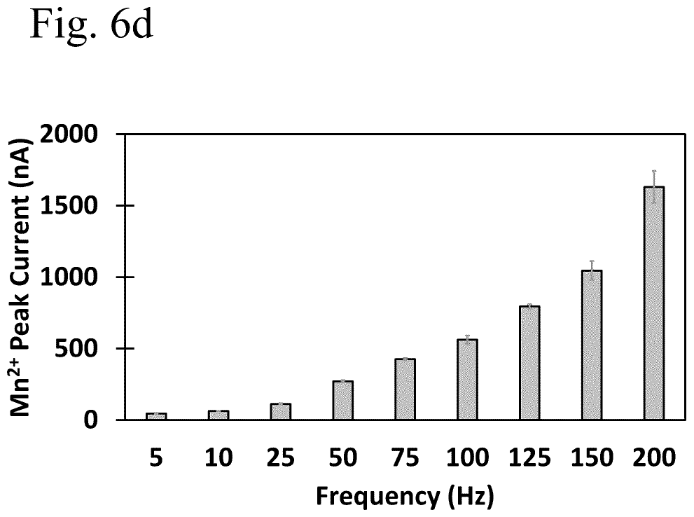

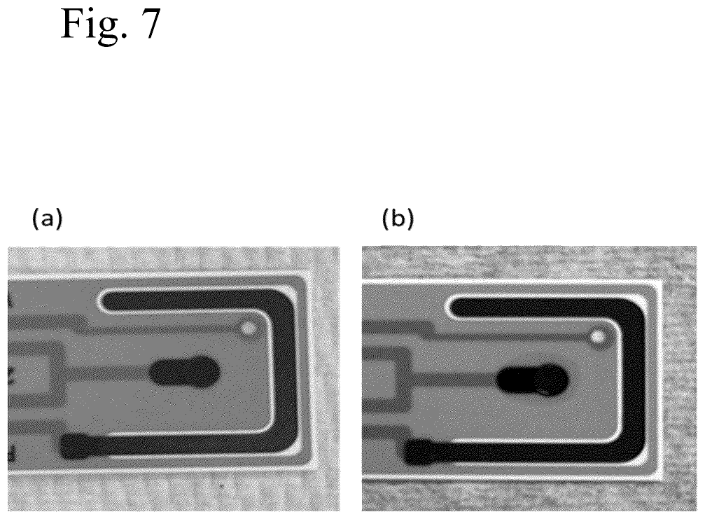

To modify the surface of a screen-printed carbon electrode (SPCE) using chitosan, a chitosan solution was prepared by dissolving 24 mg of chitosan in 20 ml of 0.1 M acetate buffer solution (AcB) at pH 4.5. The chitosan solution was then applied to the working electrode of the SPCE by drop casting 5 μL of the solution. This process was repeated four times, with each layer of chitosan solution being dried using a heat gun for 4 minutes. The resulting chitosan coating had a surface area of 7 mm². The modification process is visually illustrated in FIGS. 7(a) and 7(b) showing the SPCE before and after the chitosan coating respectively.

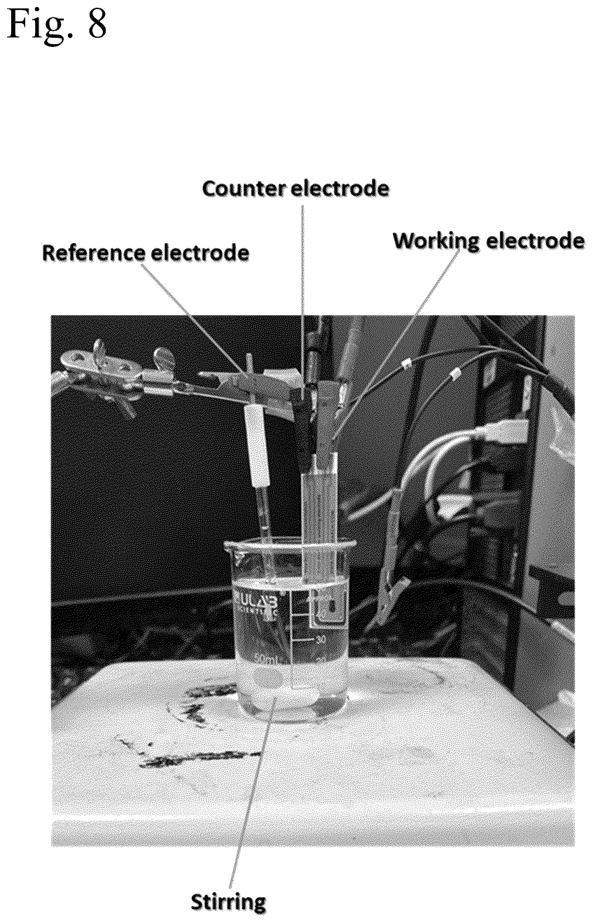

A standard stock solution of manganese ions (Mn²⁺) with a concentration of 1,000 mg/L was prepared by dissolving manganese chloride in deionized (DI) water. An acetate buffer (AcB) of 0.1 M at pH 4.5 was prepared by mixing the appropriate amount of acetic acid in DI water. Electrochemical measurements were conducted using a PalmSens4 EIS instrument. A 2 mm circular screen-printed carbon electrode (SPCE) was utilized as both the working and counter electrodes, while an A g/AgCl electrode served as the external reference electrode. Experiments were carried out in a 50 mL beaker at room temperature with continuous stirring at 240 rpm using a magnetic stir bar during the deposition step. Two types of electrodes were compared: bare SPCE and chitosan-modified SPCE. The media for all experiments was 50 ml of 0.1 M AcB at pH 4.5. Square wave adsorptive cathodic stripping voltammetry (SWAdCSV) was employed to detect trace concentrations of Mn²⁺ in the samples, using a deposition potential of +0.7 V, a deposition time of 300 seconds, an amplitude of 25 mV, and a frequency of 25 Hz. FIG. 8 shows an image of the experimental setup for the electrochemical sensor evaluation.

The MoS₂-chitosan-modified electrode was tested with a real lake water sample collected from Lake Clair in Orlando, FL. A corresponding calibration plot and correlation coefficient of Ip=0.99x−19.025 (R²=0.9923) was obtained, demonstrating the electrode's effectiveness in detecting Mn²⁺ in the lake water sample. FIG. 9 shows the corresponding calibration curve of the MoS₂-chitosan-modified electrode for Mn²⁺ detection in the lake water sample.

To further expand field applicability, Mn²⁺ detection performance was validated in surface water samples from Lake C, Lake E, and municipal tap water. Simplified calibration curves were established across low (0-20 μg/L) and high (20-100 μg/L) Mn²⁺ ranges using 95% 0.2 M AcB as the buffer. Results indicated minimal variance in sensitivity across sites, suggesting recalibration may not be required for similar water matrices.

A direct comparison between Mn²⁺ concentrations determined using site-specific versus simplified calibration curves and those measured by ICP-M S yielded recovery values within 94-107%, demonstrating high accuracy. This validates the sensor's utility for real-time monitoring across diverse environments without labor-intensive recalibration procedures.

### Optimization of Acetate Buffer Concentration

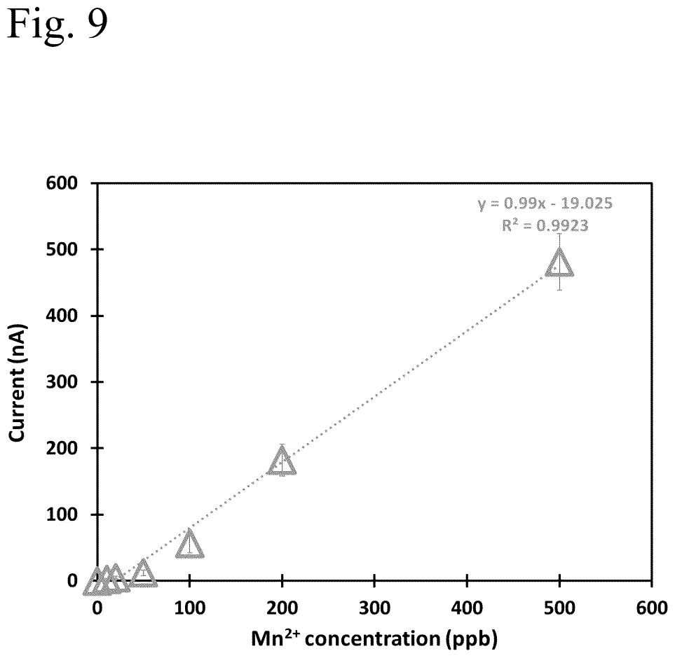

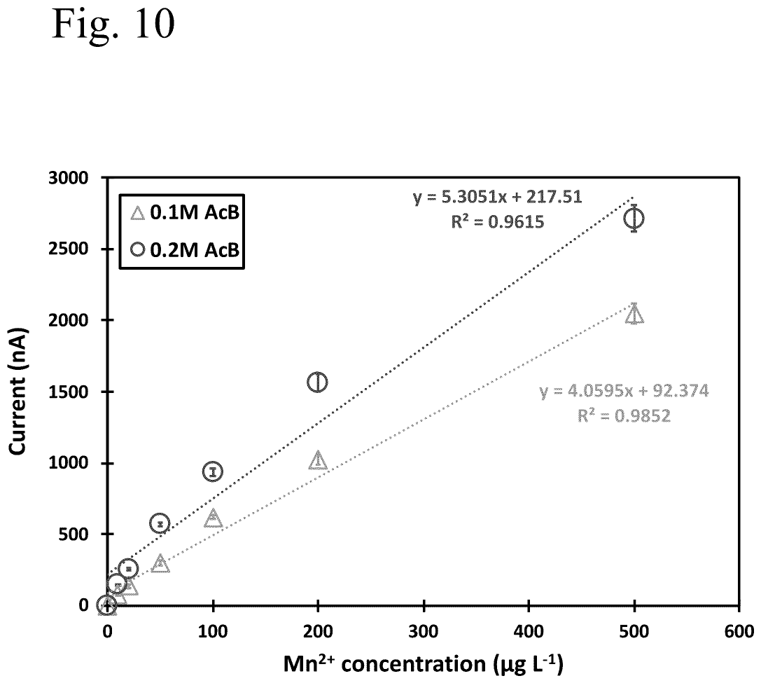

FIG. 10 illustrates the effect of acetate buffer concentration on Mn²⁺ detection sensitivity. To maximize the sensor's response, the concentration of the acetate buffer (AcB) supporting electrolyte was systematically varied. Typically, heavy metal voltammetric measurements are conducted in ˜0.1 M AcB at pH 4.5. However, increasing the AcB concentration to 0.2 M (at the same pH) notably improved the peak current for Mn²⁺, thereby enhancing the sensor's sensitivity. In experiments spanning 0 to 500 μg/L Mn²⁺, the 0.2 M AcB condition demonstrated approximately 1.31×greater slope (sensitivity) than the 0.1 M condition. Specifically, the calibration equation in 0.1 M AcB was Ip=4.0595·x+92.374 (R²=0.9852), whereas in 0.2 M AcB it was Ip=5.3051·x+217.51 (R²=0.9615). This improvement aligns with known electrochemical principles: a higher ionic-strength medium increases solution conductivity, which in turn amplifies the obtainable cathodic stripping peak currents. Thus, using a more concentrated acetate buffer (e.g., 0.2 M) as the supporting electrolyte improves Mn²⁺ quantification by boosting signal intensity and minimizing interference from extraneous ionic species in complex samples (see FIG. 10).

### Assessment of Potential Ion Interference

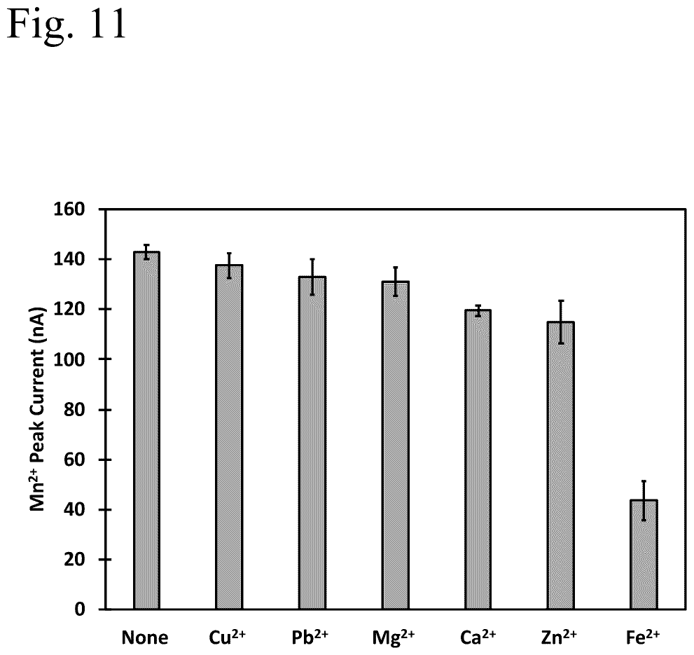

FIG. 11 presents the results of interference testing with various common cations to evaluate the TMD-biopolymer sensor's selectivity for Mn²⁺ in the presence of other metals. A series of trials was conducted where the Mn²⁺ concentration was fixed at 50 μg/L and potential interfering ions-including copper (Cu²⁺), lead (Pb²⁺), magnesium (Mg²⁺), calcium (Ca²⁺), zinc (Zn²⁺), and ferrous iron (Fe²⁺)—were each added at 50 μg/L. As shown in FIG. 11, the addition of Cu²⁺, Pb²⁺, Mg²⁺, Ca²⁺, or Zn²⁺ had negligible impact on the Mn²⁺ peak current (each causing <20% deviation from the control signal). This indicates excellent selectivity of the MoS₂-chitosan modified electrode toward Mn²⁺ over these potential interferents. In stark contrast, the presence of Fe²⁺ significantly suppressed the Mn²⁺ signal—the peak current dropped from 143±2.83 nA (Mn²⁺ alone) to 43.5±7.78 nA with Fe²⁺ present, a reduction of about 69.6%. The pronounced effect of ferrous iron is attributed to a chemical interaction: Fe²⁺ can rapidly reduce electrochemically deposited MnO₂ (formed during the stripping process) back to soluble Mn²⁺, effectively undoing the preconcentration of manganese on the electrode before it can be detected. This phenomenon, reported in prior studies of Mn²⁺ voltammetry, means Fe²⁺ competitively consumes the oxidation of Mn, leading to a lower cathodic stripping signal. Accordingly, when analyzing samples high in Fe²⁺, appropriate pretreatment or mitigation (e.g. oxidation or complexation of Fe²⁺) may be required to ensure accurate Mn²⁺ measurements. Aside from this iron-specific effect, the sensor demonstrates robust performance against a range of divalent metal background ions (consistent with FIG. 11).

### Influence of Bicarbonate Alkalinity

FIG. 12 examines how bicarbonate alkalinity—a common characteristic of natural waters, measured in terms of CaCO₃ concentration—affects Mn²⁺ detection. Alkalinity in drinking water can range roughly 20-200 mg/L as CaCO₃, and these carbonate/bicarbonate species could potentially impact the stripping voltammetry signal. In testing, Mn²⁺ peak currents were found to decrease progressively with increasing alkalinity (see FIG. 12). For example, at an alkalinity of 25 mg/L CaCO₃, the Mn²⁺ peak current dropped to about 661.5±17.68 nA from a baseline of 785.5±20.51 nA (measured with 0 mg/L CaCO₃, i.e. no added alkalinity). At higher alkalinity levels (e.g. ≥120 mg/L CaCO₃), the sensor's sensitivity suffered substantial reduction, indicating that high concentrations of bicarbonate can impede the Mn²⁺ stripping response. This decrease is likely due to pH buffering and complexation effects: in more alkaline conditions, Mn²⁺ may form insoluble hydroxides or otherwise become less electro-active, and the increased ionic strength might also alter the deposition/stripping dynamics. Despite the observed signal attenuation in very hard water, the practical impact on this sensor's utility is manageable. Given the sensor's low intrinsic detection limit (˜1.0 g/L, nearly 50× lower than the EPA's 50 μg/L secondary MCL for Mn), an analyst can simply dilute the sample (with deionized water or supporting electrolyte) to bring the effective alkalinity down to mild levels before measurement. Indeed, even a 1:1 or 1:2 dilution of a high-alkalinity sample will significantly mitigate carbonate interference while keeping Mn²⁺ within the sensor's quantifiable range. Thus, the TMD-biopolymer sensor can accurately quantify manganese in diverse water systems-including hard water—by incorporating a straightforward dilution step as needed to offset alkalinity effects (as evidenced by FIG. 12).

### Simplified Calibration Curves for Field Applications

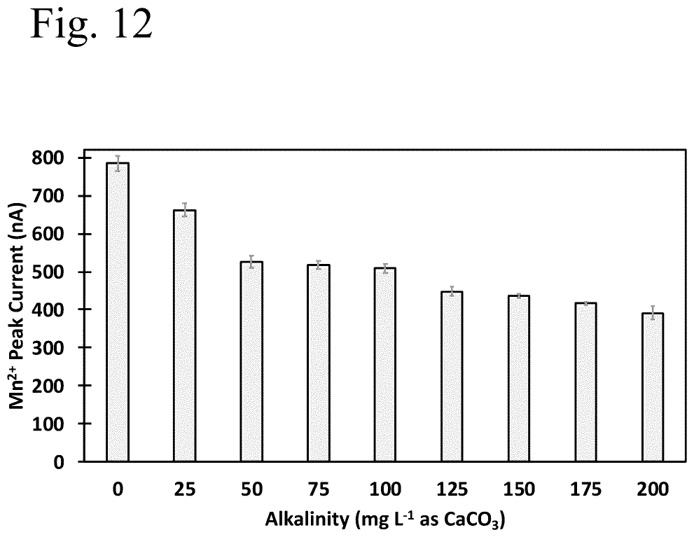

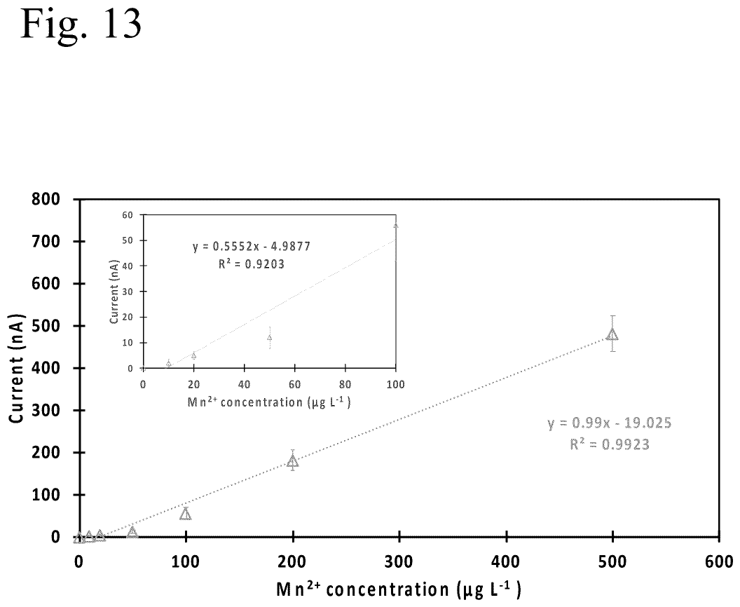

When applying the developed sensor to real-world water samples, matrix differences such as dissolved organic carbon (DOC) content and ambient ions can influence the sensor response. An initial test in a local surface water (Lake C) exemplified this: even with a high supporting buffer concentration (0.2 M AcB added), the calibration curve obtained in Lake C water showed a much lower slope compared to that in deionized (DI) water. In fact, the Mn²⁺ sensitivity in the Lake C sample (PH ˜6.8, alkalinity ˜37 mg/L as CaCO₃) was about 5.3× lower than in the DI water calibration under the same buffer conditions. This outcome (see FIG. 13, which plots a representative Mn²⁺ calibration in Lake C water diluted 1:1 with 0.2 M A cB) highlights the impact of site-specific water chemistry. It is well known that natural organic matter can bind metal ions—for instance, functional groups in DOC can chelate Mn²⁺-thereby reducing the “free” Mn²⁺ available to undergo the electrochemical deposition/stripping process. Such matrix effects can markedly diminish sensor sensitivity in some waters. Traditionally, one would address this by generating site-specific calibration curves for each water source and periodically recalibrating the sensor for accuracy. However, that approach is time-consuming and impractical for frequent field monitoring across multiple sites.

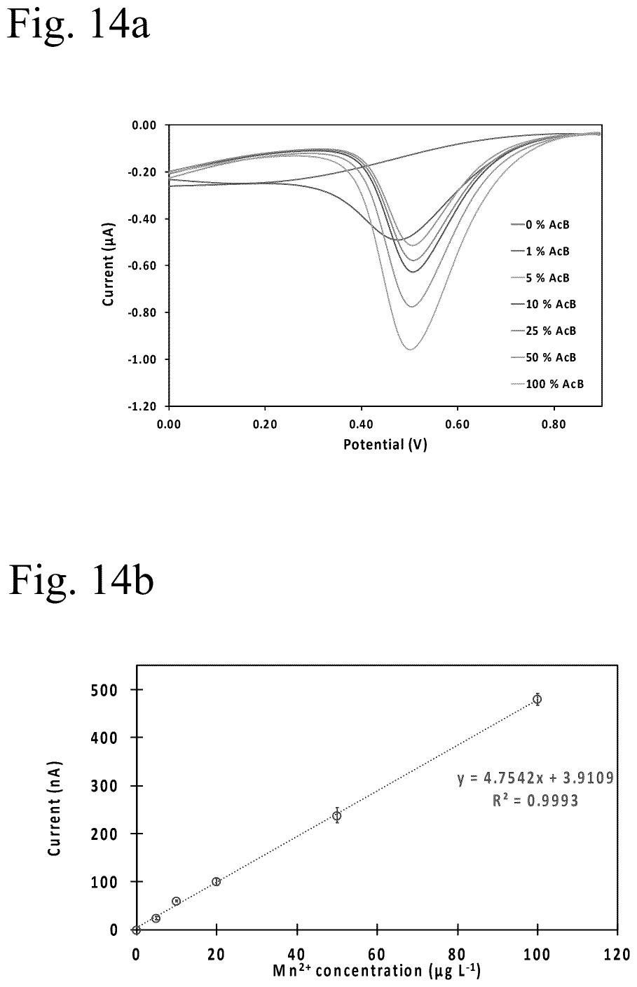

To overcome this challenge, the present invention employs a streamlined calibration strategy involving partial sample dilution with the acetate buffer to normalize matrix differences. In other words, rather than recalibrating for every new water type, the sample itself is conditioned in a standardized way so that a single calibration curve can be applied broadly. Experiments were conducted to determine how much buffer dilution is required to reliably measure Mn²⁺ in various waters. FIG. 14(a) shows square-wave adsorptive cathodic stripping voltammograms for a 50 μg/L Mn²⁺ spike in Lake C water under different buffer dilution ratios (ranging from 0% up to 100% addition of 0.2 M AcB). The data indicate that with at least a 5% addition of the 0.2 M acetate buffer (i.e., using 95% sample +5% buffer, equivalent to a 1:20 dilution), a clear and analytically useful Mn²⁺ peak is obtained. Little additional benefit was observed beyond the 5-10% buffer level, meaning a relatively small volume of buffer is sufficient to counteract the lake matrix effects. Based on these findings, a 5% (v/v) 0.2 M AcB dilution was adopted as a standard preparation for real samples. Using this approach, FIG. 14(b) illustrates a calibration curve for Mn²⁺ detection in the Lake C water sample with 5% buffer added. The slope and linearity of this curve in the lake matrix (with partial buffering) are markedly improved over the undiluted case, closely approaching those of the DI water calibration. This demonstrates that a modest buffer dilution can effectively restore sensor sensitivity in complex samples without the need for exhaustive individual recalibrations.

TABLE 2
Water sample characteristics obtained from different sources

| | | Alkalinity | DOC |
|---|---|---|---|
| Water matrix | pH | (mg L⁻¹) | (mg L⁻¹) |
| Tap Water | 8.0 ± 0.15 | 60.5 ± 0.39 | 1.96 ± 0.1 |
| Lake C | 7.0 ± 0.06 | 20.8 ± 0.40 | 11.19 ± 0.2 |
| Lake E | 6.9 ± 0.08 | 4.3 ± 0.14 | 5.37 ± 0.1 |

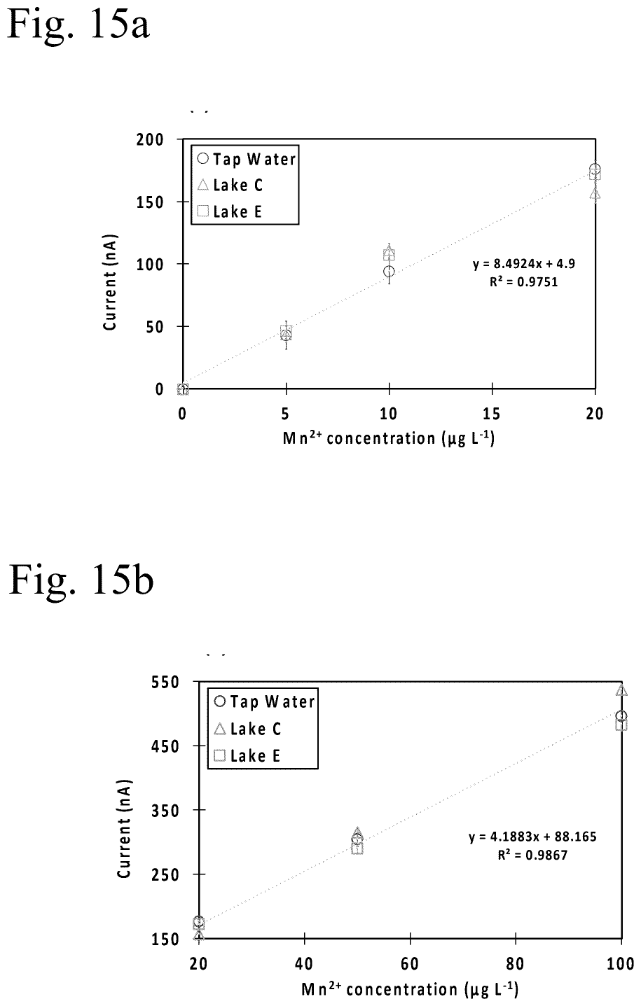

Building on the Lake C results, FIG. 15 presents the consolidated “simplified” calibration curves derived from multiple real-water sources under the standardized buffer dilution protocol. In this evaluation, two lake samples (Lake C and Lake E) and a municipal tap water sample were each diluted with 0.2 M AcB to ˜95% buffer (5% sample), and the sensor response was measured over a range of Mn²⁺ concentrations. The data from all three water sources were then combined to generate unified calibration plots for two concentration ranges: low (0-20 μg/L) and high (20-100 μg/L). FIG. 15(a) shows the composite calibration for the 0-20 μg/L range, and FIG. 15(b) shows the 20-100 μg/L range. Impressively, both plots exhibit strong linearity and overlap of data points from the different water matrices. The fitted equations—for example, Ip=8.4924·x+4.9 (R²=0.9751) for the low-range calibration and/p=4.1883·x+88.165 (R²=0.9867) for the high-range calibration-indicate that one calibration curve can indeed serve for all tested waters under these buffered conditions. In practical terms, this means the sensor output in a partially buffered lake or tap water sample can be interpreted using a single master calibration, avoiding the need for creating separate curves for each source. The use of 95% 0.2 M AcB as a diluent effectively standardized the chemical environment, so the TMD-biopolymer-coated electrode responded to Mn²⁺ consistently across otherwise different water chemistries (see FIGS. 15(a) and 15(b)). This simplified calibration approach greatly enhances field deployability, as it reduces maintenance and recalibration efforts.

### Real Water Sample Applications and Validations

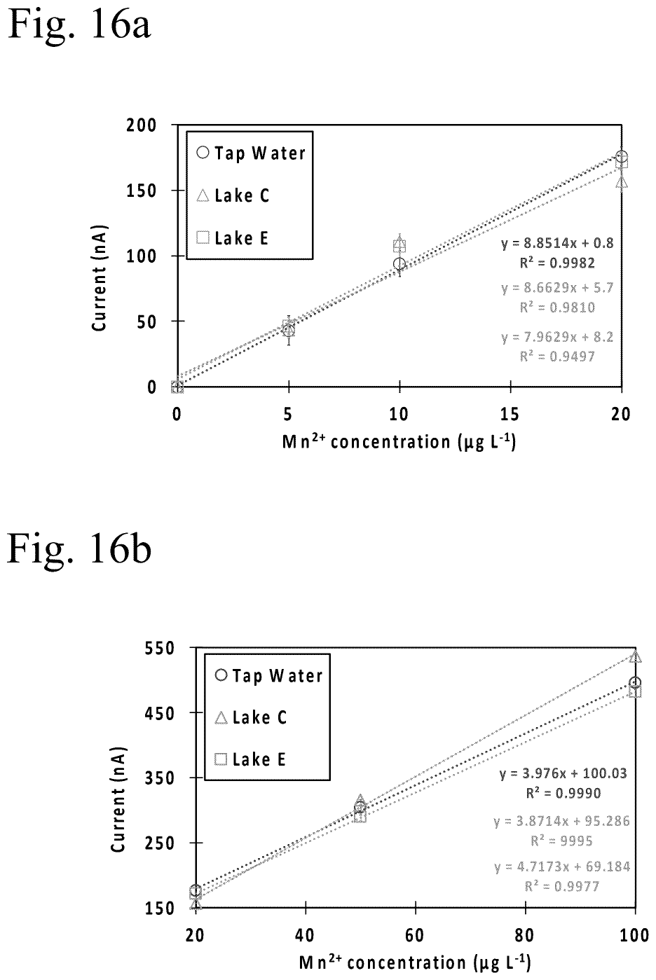

To validate the accuracy of the simplified calibration approach, the sensor was tested on actual environmental samples with known manganese additions and compared against laboratory analysis. Water samples from two different lakes (Lake C and Lake E) and a city tap water supply were spiked with a known concentration of Mn²⁺ (approximately 70-80 μg/L, in the mid-high range of the sensor) and then measured using the TMD-biopolymer sensor (with the 5% buffer calibration) versus by ICP-M S as an authoritative reference. In addition, for each sample, a “site-specific” calibration curve was separately constructed (100% of that sample with buffer, no mixing of data between sources) to see if any significant discrepancies remained. FIG. 16(a) and FIG. 16(b) plot the individual calibration curves for each water source in the low (0-20 μg/L) and high (20-100 μg/L) ranges, respectively, after applying the 95% buffer dilution. As shown in FIG. 16, the three curves (tap, Lake C, Lake E) are very close to one another, with minimal divergence in slope or intercept across the different water matrices. This confirms that the 20-fold dilution effectively minimized site-specific matrix effects: under these conditions, any two given water sources yield nearly overlapping calibration lines for Mn²⁺.

TABLE 3
Mn²⁺ measurements by TMD-biopolymer-coated
SPCE sensor using the simplified calibration curve

| | Mn²⁺ | Mn²⁺ detected | | Mn²⁺ detected | |
|---|---|---|---|---|---|
| | detected | by site-specific | Mn²⁺ | by simplified | Mn²⁺ |
| Water | by ICP-MS | calibration | Recovery | calibration | Recovery |
| matrix | (μg L⁻¹) | curve (μg L⁻¹) | (%) | curve (μg L⁻¹) | (%) |
| Tap Water | 79.25 ± 0.05 | 75.10 ± 3.97 | 94.76 ± 0.21 | 77.07 ± 0.05 | 97.25 ± 0.23 |
| Lake C | 70.63 ± 0.50 | 68.66 ± 3.44 | 97.21 ± 0.61 | 75.69 ± 0.50 | 107.16 ± 0.60 |
| Lake E | 69.80 ± 0.79 | 67.01 ± 3.62 | 96.00 ± 0.72 | 65.63 ± 0.79 | 94.02 ± 0.74 |

Consistent with this observation, the performance of the unified calibration was quantified in terms of recovery of the spiked Mn²⁺. The sensor's readings, when interpreted via the simplified (combined) calibration, achieved recovery rates of about 94.76-97.25% for the tap water sample, 94.02-96.00% for the Lake E sample, and 97.21-107.16% for the Lake C sample (the range in each case reflecting the slight difference between using the master calibration vs. the individual curve). All of these values are within acceptable accuracy ranges (approximately 90-110% recovery is typical for field sensors, and even 107% is within a 70-120% tolerance often allowed in analytical methods). The high recoveries obtained across all three distinct water sources demonstrate the robustness and reliability of the TMD-biopolymer-coated SPCE sensor in diverse environmental conditions. In essence, the sensor-when used with the described buffer-dilution calibration technique—can accurately detect and quantify Mn²⁺ in different real-world waters without needing frequent recalibration or specialized adjustments for each site. Consequently, the invention provides an effective and practical alternative to traditional lab-bound M n analysis, suitable for on-site, rapid monitoring of manganese in drinking water supplies and natural waters. The added experimental data (FIGS. 10-16b) verify that the sensor design and methodology maintain high performance even in complex matrices, further supporting the broad utility of the invention in field applications.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

### Glossary of Claim Terms

Acetate buffer means an aqueous solution composed of acetic acid and its conjugate base, acetate, typically used to stabilize and maintain the pH of solutions during electrochemical processes. In the context of the claimed invention, the acetate buffer solution specifically maintains a pH of about 4.5, ensuring optimal electrochemical conditions for manganese ion deposition onto a screen-printed carbon electrode (SPCE). This stable pH condition promotes the efficient formation of a molybdenum disulfide (MoS₂) and chitosan composite, critical to the accurate electrochemical detection of manganese ions using square wave adsorptive cathodic stripping voltammetry (SWAdCSV). Additionally, acetate buffer enhances the reproducibility of electrochemical measurements by preventing significant fluctuations in pH during the deposition and detection stages, thereby ensuring reliable, consistent manganese ion quantification across various water samples and operational scenarios.

Amplitude means the magnitude of the voltage difference applied during the square wave voltammetry (SWV) measurement process. It is a parameter that influences the sensitivity and resolution of the detection method. For this invention, amplitudes in the range of 5 to 150 mV are evaluated to optimize the detection of manganese ions (Mn²⁺) in water samples. The selected amplitude affects the peak current observed during the voltammetry measurement, impacting the calibration and quantification of Mn²⁺ concentrations. The optimal amplitude is useful for ensuring accurate and sensitive detection of the target ions.

Bicarbonate means a polyatomic anion characterized by the chemical formula HCO₃⁻, typically acting as a buffering agent in natural waters, impacting metal speciation and reactivity. Within the context of electrochemical manganese ion detection, bicarbonate influences the equilibrium between free-state manganese ions and DOC-bound manganese. The presence of bicarbonate can affect electrochemical responses during square wave adsorptive cathodic stripping voltammetry (SWAdCSV), thus understanding its impact is essential to accurately quantify manganese ions using the claimed sensor. The influence of bicarbonate on manganese ion behavior underscores the necessity of accounting for such chemical interactions during the calibration and application of electrochemical sensors in natural or treated water sources, ensuring precise measurement and effective environmental monitoring.

Calibration curves means graphical representations that relate measured electrochemical signals to known concentrations of an analyte, providing a means to quantify unknown concentrations in samples. In the context of this invention, calibration curves are generated using square wave adsorptive cathodic stripping voltammetry (SWAdCSV) signals obtained from solutions of known manganese ion concentrations. These curves are critical for establishing a quantitative relationship between the peak currents produced at the working electrode and the manganese ion concentration in water. Reliable calibration curves ensure the sensor's accuracy and precision, allowing for the detection and quantification of manganese ions within regulatory limits and ensuring that sensor performance remains consistent across various environmental conditions and operational settings.

Chitosan means a natural, low-cost biopolymer derived from the partial deacetylation of chitin, which is found in the exoskeletons of crustaceans such as crabs and shrimp. It is characterized by its cationic properties and biocompatibility, making it useful in various applications, including water treatment and sensor development. In this invention, chitosan is utilized for its ability to adsorb manganese ions (Mn²⁺), enhancing the sensitivity and selectivity of the electrochemical sensor. The chitosan is prepared by dissolving it in an acetate buffer solution, which is then deposited onto a screen-printed carbon electrode (SPCE) to form a composite material with molybdenum disulfide (MoS₂).

Composite solution means a mixture of transition metal dichalcogenide (TMD) and chitosan solutions prepared for deposition onto the electrode. In this invention, the TMD solution is prepared by dissolving TMD powder, such as molybdenum disulfide (MoS₂), in isopropyl alcohol. The chitosan solution is prepared by dissolving chitosan in an acetate buffer. These solutions are mixed in specific proportions to form the composite solution, which is then used to coat the working electrode. The composite solution enhances the electrode's ability to detect manganese ions (Mn²⁺) in water samples by combining the catalytic properties of TMD with the adsorption capabilities of chitosan.

Counter electrode means an electrode used to complete the electrical circuit in the electrochemical cell. It works in conjunction with the working electrode and the reference electrode to facilitate the redox reactions necessary for the detection of analytes. In this invention, the counter electrode is part of a three-electrode system, where it helps to control the current flow during the electrochemical measurements. The counter electrode is typically made of an inert material, such as platinum or carbon, and its primary function is to balance the current at the working electrode to maintain a stable and controlled electrochemical environment.

Deposition means the process of coating the working electrode with the TMD-chitosan composite solution. This process involves applying a thin layer of the composite material onto the electrode surface to enhance its electrochemical properties. In this invention, the deposition is achieved using an electrochemical method, where the TMD-chitosan composite solution is applied to the screen-printed carbon electrode (SPCE) under controlled conditions. The deposition parameters, such as potential and time, are optimized to ensure uniform coating and effective interaction of the composite material with manganese ions (Mn²⁺) in the water samples. The deposition process is important for improving the sensitivity and selectivity of the electrochemical sensor.

DOC-Bound means manganese ions complexed with dissolved organic carbon (DOC) present in environmental water samples. DOC-bound manganese represents a chemically distinct form from the free-state manganese ions and can significantly impact electrochemical detection by altering the electrochemical behavior and response of manganese during the stripping process. Understanding and differentiating DOC-bound manganese is essential for accurate electrochemical quantification of free-state manganese ions using the claimed sensor. The ability of the sensor to discriminate between these forms ensures precise environmental monitoring and regulatory compliance, providing a clear understanding of manganese ion availability, bioavailability, and potential ecological impacts in various water sources.

Electrochemical deposition means a fabrication technique wherein materials are deposited onto conductive substrates using an applied electrical potential, causing reduction or oxidation reactions that result in a uniform coating. In the claimed invention, electrochemical deposition is employed to form a composite layer of molybdenum disulfide (MoS₂) and chitosan on the surface of screen-printed carbon electrodes (SPCEs). Parameters such as deposition potential (+0.7 V to +1.1 V) and duration (30 to 900 seconds) are specifically optimized to achieve uniform coatings that exhibit high sensitivity and reproducibility for manganese ion detection. This deposition method ensures controlled formation of sensor layers that maximize the electrochemical interactions required for effective manganese ion detection using square wave adsorptive cathodic stripping voltammetry (SWA dCSV).

Free-state means manganese ions existing in water in their dissolved ionic form, unbound by dissolved organic carbon (DOC) or other complexing agents. Free-state manganese ions are directly available for electrochemical detection and measurement, contrasting with complexed or DOC-bound forms. Accurate detection of free-state manganese ions is particularly crucial as it directly correlates to potential environmental and human health impacts. The claimed sensor specifically targets free-state manganese ions through its molybdenum disulfide (MoS₂) and chitosan composite coating, ensuring precise quantification and monitoring. Understanding free-state ion behavior is essential for effective water quality management, regulatory compliance, and assessing ecological and health risks associated with manganese contamination.

Limit of detection (LOD) means the lowest concentration of manganese ions (Mn²⁺) that can be reliably detected by the sensor. It is a parameter that defines the sensitivity of the electrochemical sensor. The LOD is determined by analyzing the sensor's response to decreasing concentrations of Mn²⁺ and identifying the minimum concentration at which the sensor can still produce a measurable signal distinguishable from the background noise. In this invention, the LOD for the TMD-chitosan-modified sensor is found to be significantly lower than that of a bare SPCE, indicating the enhanced sensitivity achieved through the use of the composite material. The LOD is important for applications requiring the detection of trace levels of Mn²⁺ in water samples.

Manganese ions (Mn²⁺) means divalent manganese ions present in water samples. Manganese is an essential trace element for human health, but its excessive presence in water can pose health risks. In this invention, the focus is on detecting Mn²⁺ in water samples using an electrochemical sensor. Mn²⁺ ions are targeted due to their common occurrence in environmental water sources and the potential health implications of their elevated levels. The electrochemical sensor developed in this invention utilizes the TMD-chitosan composite to selectively and sensitively detect Mn²⁺, providing a reliable method for monitoring manganese concentrations in various water samples, including drinking water and natural water bodies.

Molybdenum disulfide (MoS₂) means a specific type of transition metal dichalcogenide used in the sensor. MoS₂ is characterized by its layered structure, where molybdenum atoms are sandwiched between layers of sulfur atoms. This structure gives MoS₂ unique electronic, catalytic, and mechanical properties, making it suitable for use in electrochemical applications. In this invention, MoS₂ is combined with chitosan to form a composite material that enhances the sensitivity and selectivity of the electrochemical sensor for detecting manganese ions (Mn²⁺) in water samples. The MoS₂ provides catalytic activity, facilitating the electrochemical reactions necessary for the detection process.

Partial deacetylation means a chemical process involving the selective removal of acetyl groups from chitin, typically obtained from crustacean shells, resulting in the formation of chitosan. The degree of deacetylation significantly influences the physical, chemical, and biological properties of the resulting chitosan, such as its solubility, viscosity, film-forming capabilities, and metal-binding affinity. In the claimed invention, chitosan with an optimal degree of partial deacetylation is employed to create composite films with molybdenum disulfide (MoS₂). This specific degree of deacetylation ensures effective metal ion chelation, stable film deposition, and uniform composite formation on screen-printed carbon electrodes (SPCEs), enhancing the electrochemical sensor's overall sensitivity and reproducibility in detecting manganese ions via square wave adsorptive cathodic stripping voltammetry (SWA dCSV).

Reference electrode means an electrode with a stable and known potential, used for accurate measurement in electrochemical cells. It serves as a constant reference point against which the potential of the working electrode can be measured. In this invention, an A g/AgCl electrode is used as the reference electrode. The stability and reliability of the reference electrode are crucial for obtaining precise and reproducible electrochemical measurements. The reference electrode helps to maintain a consistent electrochemical environment, allowing for the accurate detection of manganese ions (Mn²⁺) in water samples by providing a stable baseline potential for the working electrode.

Screen-printed carbon electrode (SPCE) means a carbon-based electrode prepared using screen-printing techniques. SPCEs are commonly used in electrochemical sensors due to their low cost, ease of fabrication, and versatility. In this invention, the SPCE serves as the substrate for the working electrode, onto which the TMD-chitosan composite is deposited. The screen-printing process allows for the production of electrodes with consistent dimensions and properties, contributing to the reproducibility of the sensor's performance. SPCEs are particularly suitable for developing portable and disposable electrochemical sensors for environmental monitoring applications, including the detection of manganese ions (Mn²⁺) in water samples.

Square wave adsorptive cathodic stripping voltammetry (SWAdCSV) means an electrochemical technique used for sensitive detection of manganese ions (Mn²⁺) by applying square wave voltages. SWA dCSV involves applying a series of potential steps, with each step consisting of a forward and reverse pulse, to the working electrode while monitoring the resulting current. This technique enhances the sensitivity and selectivity of the sensor by concentrating the analyte onto the electrode surface before the stripping step. In this invention, SWAdCSV is used to detect trace concentrations of Mn²⁺ in water samples. The optimized parameters for SWAdCSV, such as deposition potential, deposition time, amplitude, and frequency, are crucial for achieving accurate and sensitive detection of Mn²⁺.

Transition metal dichalcogenide (TMD) means a class of materials including molybdenum disulfide (MoS₂) used for their catalytic properties. TMDs have a layered structure, with metal atoms sandwiched between layers of chalcogen atoms, resulting in unique electronic, optical, and catalytic properties. In this invention, TMDs such as MoS₂ are combined with chitosan to form a composite material that enhances the performance of the electrochemical sensor. The TMD component provides catalytic activity, improving the sensor's ability to detect manganese ions (Mn²⁺) in water samples. The use of TMDs in the composite material contributes to the sensitivity and selectivity of the sensor.

Working electrode means the electrode on which the electrochemical reaction occurs, coated with the TMD-chitosan composite. The working electrode is the primary site for the redox reactions involved in the detection of analytes. In this invention, the working electrode is a screen-printed carbon electrode (SPCE) coated with a composite of transition metal dichalcogenide (TMD) and chitosan. The TMD-chitosan coating enhances the electrode's sensitivity and selectivity for detecting manganese ions (Mn²⁺) in water samples. The working electrode interacts with the Mn²⁺ ions in the sample.

### INCORPORATION BY REFERENCE

[1] E. Boselli, Z. Wu, A. Friedman, B. Claus Henn, I. Papautsky, Validation of electrochemical sensor for determination of manganese in drinking water, Environmental Science & Technology, 55 (2021) 7501-9.
[2] S. Sarkar, E. Malovic, D. S. Harischandra, H. A. Ngwa, A. Ghosh, C. Hogan, et al., Manganese exposure induces neuroinflammation by impairing mitochondrial dynamics in astrocytes, Neurotoxicology, 64 (2018) 204-18.
[3] W. H. Organization, Manganese in drinking water: background document for development of WHO guidelines for drinking-water quality, World Health Organization2021.
[4] A. P. Sanders, T. A. Desrosiers, J. L. Warren, A. H. Herring, D. Enright, A. F. Olshan, et al., Association between arsenic, cadmium, manganese, and lead levels in private wells and birth defects prevalence in North Carolina: a semi-ecologic study, B M C public health, 14 (2014) 1-12.
[5] Y. Shilina, B. Ziv, A. Meir, A. Banerjee, S. Ruthstein, S. Luski, et al., Combined electron paramagnetic resonance and atomic absorption spectroscopy/inductively coupled plasma analysis as diagnostics for soluble manganese species from M n-based positive electrode materials in Li-ion cells, Analytical chemistry, 88 (2016) 4440-7.
[6] M. R. Pourjavid, M. Arabieh, S. R. Yousefi, A. A. Sehat, Interference free and fast determination of manganese (II), iron (III) and copper (II) ions in different real samples by flame atomic absorption spectroscopy after column graphene oxide-based solid phase extraction, Microchemical Journal, 129 (2016) 259-67.
[7] K. Wróbel, K. Wróbel, E. M. C. Urbina, Determination of total aluminum, chromium, copper, iron, manganese, and nickel and their fractions leached to the infusions of black tea, green tea, Hibiscus sabdariffa, and llex paraguariensis (mate) by ETA-AAS, Biological Trace Element Research, 78 (2000) 271-80.
[8] J. A. Nunes, B. L. Batista, J. L. Rodrigues, N. M. Caldas, J. A. Neto, F. Barbosa Jr, A simple method based on ICP-MS for estimation of background levels of arsenic, cadmium, copper, manganese, nickel, lead, and selenium in blood of the Brazilian population, Journal of Toxicology and Environmental Health, Part A, 73 (2010) 878-87.
[9] W. Kang, X. Pei, A. Bange, E. N. Haynes, W. R. Heineman, I. Papautsky, Copper-based electrochemical sensor with palladium electrode for cathodic stripping voltammetry of manganese, Analytical chemistry, 86 (2014) 12070-7.
[10] T.-H. Lu, J.-F. Huang, I.-W. Sun, Perfluorinated anion-exchange polymer mercury film electrode for anodic stripping voltammetric determination of zinc (II): effect of model organic compounds, Analytica chimica acta, 454 (2002) 93-100.
[11] M. Pavličková, L. Lorencová, M. Hatala, M. Kováč, J. Tkáč, P. Gemeiner, Facile fabrication of screen-printed M oS2 electrodes for electrochemical sensing of dopamine, Scientific Reports, 12 (2022) 11900.
[12] A. Y in, X. Wei, Y. Cao, H. Li, High-quality molybdenum disulfide nanosheets with 3D structure for electrochemical sensing, Applied Surface Science, 385 (2016) 63-71.
[13] M. Chhowalla, H. S. Shin, G. Eda, L.-J. Li, K. P. Loh, H. Zhang, The chemistry of two-dimensional layered transition metal dichalcogenide nanosheets, Nature chemistry, 5 (2013) 263-75.
[14] J.-H. Hwang, M. A. Islam, H. Choi, T.-J. Ko, K. L. Rodriguez, H.-S. Chung, et al., Improving Electrochemical Pb²⁺ Detection Using a Vertically Aligned 2D MoS₂ Nanofilm, Analytical chemistry, 91 (2019) 11770-7.
[15] J. Chen, N. Kuriyama, H. Yuan, H. T. Takeshita, T. Sakai, Electrochemical hydrogen storage in Mos2 nanotubes, Journal of the American Chemical Society, 123 (2001) 11813-4.
[16] P. Raybaud, J. Hafner, G. Kresse, S. Kasztelan, H. Toulhoat, Structure, energetics, and electronic properties of the surface of a promoted MoS₂ catalyst: an ab initio local density functional study, Journal of Catalysis, 190 (2000) 128-43.
[17] E. Parzinger, B. Miller, B. Blaschke, J. A. Garrido, J. W. A ger, A. Holleitner, et al., Photocatalytic stability of single—and few-layer MoS₂, A CS nano, 9 (2015) 11302-9.
[18] K.-J. Huang, L. Wang, J. Li, Y.-M. Liu, Electrochemical sensing based on layered MoS2-graphene composites, Sensors and Actuators B: Chemical, 178 (2013) 671-7.
[19] A. Sinha, B. Tan, Y. Huang, H. Zhao, X. Dang, J. Chen, et al., M oS2 nanostructures for electrochemical sensing of multidisciplinary targets: a review, TrAC Trends in Analytical Chemistry, 102 (2018) 75-90.
[20] Z. Wang, B. Mi, Environmental Applications of 2D Molybdenum Disulfide (MoS₂) Nanosheets, Environmental Science & Technology, 51 (2017) 8229-44.
[21] J. Yang, J.-H. Yu, J. R. Strickler, W.-J. Chang, S. Gunasekaran, Nickel nanoparticle-chitosan-reduced graphene oxide-modified screen-printed electrodes for enzyme-free glucose sensing in portable microfluidic devices, Biosensors and Bioelectronics, 47 (2013) 530-8.
[22] S. Yang, X. Liu, X. Zeng, B. Xia, J. Gu, S. Luo, et al., Fabrication of nano-copper/carbon nanotubes/chitosan film by one-step electrodeposition and its sensitive determination of nitrite, Sensors and Actuators B: Chemical, 145 (2010) 762-8.
[23] J.-H. Hwang, X. Wang, P. Pathak, M. M. Rex, H. J. Cho, W. H. Lee, Enhanced electrochemical detection of multiheavy metal ions using a biopolymer-coated planar carbon electrode, leee T Instrum M eas, 68 (2019) 2387-93.
[24] S. Jawaheer, S. White, S. Rughooputh, D. C. Cullen, Development of a common biosensor format for an enzyme based biosensor array to monitor fruit quality, Biosensors and Bioelectronics, 18 (2003) 1429-37.
[25] J.-H. Hwang, P. Pathak, X. Wang, K. L. Rodriguez, H. J. Cho, W. H. Lee, A Novel Bismuth-Chitosan Nanocomposite Sensor for Simultaneous Detection of Pb (II), Cd (II) and Zn (II) in Wastewater, Micromachines, 10 (2019) 511.
[26] J.-H. Hwang, P. Pathak, X. Wang, K. L. Rodriguez, J. Park, H. J. Cho, et al., A novel Fe-Chitosan-coated carbon electrode sensor for in situ As (III) detection in mining wastewater and soil leachate, Sensors and Actuators B: Chemical, 294 (2019) 89-97.
[27] W. Kang, Point-of-care Sensors for Determination of Manganese in Clinical Applications: University of Cincinnati; 2016.
[28] J.-H. Hwang, S. Fahad, H. Ryu, K. L. Rodriguez, J. Santo Domingo, A. Kushima, et al., Recycling urine for bioelectrochemical hydrogen production using a M oS2 nano carbon coated electrode in a microbial electrolysis cell, J Power Sources, 527 (2022) 231209.
[29] S. Saha, P. Sarkar, Differential pulse anodic stripping voltammetry for detection of As (III) by Chitosan-Fe (OH) 3 modified glassy carbon electrode: A new approach towards speciation of arsenic, Talanta, 158 (2016) 235-45.
[30] S. Jiang, R. Cheng, R. Ng, Y. Huang, X. Duan, Highly sensitive detection of mercury (II) ions with few-layer molybdenum disulfide, Nano Research, 8 (2015) 257-62.
[31] M. Chigane, M. Ishikawa, Manganese oxide thin film preparation by potentiostatic electrolyses and electrochromism, Journal of the Electrochemical Society, 147 (2000) 2246.
[32] J. Xi, X. Huang, M. Hu, W. Xiang, Dependence of laser parameters on structural properties of pulsed laser-deposited MoS 2 thin films applicable for field effect transistors, Journal of Materials Science: Materials in Electronics, 31 (2020) 21118-27.
[33] H. Ji, S. Hu, S. Shi, B. Guo, W. Hou, G. Y ang, Rapid microwave-hydrothermal preparation of few-layer M oS 2/C nanocomposite as anode for highly reversible lithium storage properties, Journal of Materials Science, 53 (2018) 14548-58.
[34] R. Tholkappiyan, A. N. Naveen, K. Vishista, F. Hamed, Investigation on the electrochemical performance of hausmannite Mn³⁰⁴ nanoparticles by ultrasonic irradiation assisted co-precipitation method for supercapacitor electrodes, Journal of Taibah University for Science, 12 (2018) 669-77.
[35] A. Ghods, C. Zhou, I. T. Ferguson, Structural and optical properties of (Zn, Mn) O thin films prepared by atomic layer deposition, Journal of Vacuum Science & Technology A, 38 (2020).
[36] M. C. Biesinger, B. P. Payne, A. P. Grosvenor, L. W. Lau, A. R. Gerson, R. S. C. Smart, Resolving surface chemical states in X PS analysis of first row transition metals, oxides and hydroxides: Cr, Mn, Fe, Co and Ni, Applied Surface Science, 257 (2011) 2717-30.
[37] C. A. Rusinek, A. Bange, M. Warren, W. Kang, K. Nahan, I. Papautsky, et al., Bare and polymer-coated indium tin oxide as working electrodes for manganese cathodic stripping voltammetry, Analytical chemistry, 88 (2016) 4221-8.
[38] N. Koper, L. Leston, T. M. Baker, C. Curry, P. Rosa, Effects of ambient noise on detectability and localization of avian songs and tones by observers in grasslands, Ecology and evolution, 6 (2016) 245-55

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

It is also to be understood that the following claims are intended to cover all of the generic and specific features of the invention herein described, and all statements of the scope of the invention which, as a matter of language, might be said to fall therebetween.

What is claimed is:

1. An electrochemical sensor composite for manganese ion detection consisting essentially of molybdenum disulfide (MoS₂) and chitosan deposited directly onto a screen-printed carbon electrode (SPCE).

2. The sensor composite of claim 1, wherein MoS₂ is present as exfoliated nanosheets.

3. The sensor composite of claim 1, wherein the chitosan is derived from partial deacetylation of chitin.

4. The sensor composite of claim 1, wherein the MoS₂ and chitosan are combined in a solution containing isopropyl alcohol and acetate buffer prior to deposition.

5. The sensor composite of claim 4, wherein the acetate buffer maintains a pH of 4.5.

6. The sensor composite of claim 1, deposited onto the SPCE via electrochemical deposition at a potential between +0.7 V to +1.1 V for 30 to 900 seconds.

7. The sensor composite of claim 1, wherein the deposition surface area on the SPCE is approximately 7 mm².

8. The sensor composite of claim 1, wherein the composite excludes graphene and carbon nanotubes.

9. An electrochemical sensing apparatus for detecting manganese ions comprising:
a. a screen-printed carbon electrode (SPCE);
b. a working electrode coated with a composite of molybdenum disulfide (MoS₂) and chitosan;
c. a reference electrode configured to maintain a stable potential;
d. a counter electrode to complete an electrical circuit; and
e. an electrochemical analyzer configured specifically for performing square wave adsorptive cathodic stripping voltammetry (SWA dCSV).

10. The apparatus of claim 9, wherein the electrochemical analyzer operates with an amplitude between 5 and 150 mV and a frequency between 5 and 200 Hz.

11. The apparatus of claim 9, wherein the composite is electrochemically deposited onto the working electrode at a potential between +0.7 V and +1.1 V for a time between 30 and 900 seconds.

12. The apparatus of claim 9, wherein the reference electrode is an A g/AgCl electrode.

13. The apparatus of claim 9, configured for portable, field-deployable use.

14. The apparatus of claim 9, integrated into a disposable sensor strip.

15. The apparatus of claim 9, wherein no additional chemical ligand is required for manganese ion accumulation.

16. A method for detecting manganese ions in water, comprising:
a. preparing a composite solution comprising molybdenum disulfide (MoS₂) dissolved in isopropyl alcohol and chitosan dissolved in acetate buffer;
b. electrodepositing the composite solution onto a screen-printed carbon electrode (SPCE);
c. immersing the electrode in a water sample;
d. applying a deposition potential of between +0.7 V and +1.1 V for between 30 and 900 seconds; and
e. performing square wave adsorptive cathodic stripping voltammetry (SWAdCSV) to detect manganese ions.

17. The method of claim 16, wherein the SWA dCSV is performed at an amplitude of between 5 and 150 mV and a frequency between 5 and 200 Hz.

18. The method of claim 16, further comprising adjusting the water sample pH to approximately 4.5 with acetate buffer prior to step (c).

19. The method of claim 16, wherein the electrodeposition step (b) is performed at +0.9 V.

20. The method of claim 16, wherein the electrode surface area coated is about 7 mm².

21. The method of claim 16, wherein the electrode is reused after a regeneration step applying a cleaning potential.

22. The method of claim 16, wherein the composite is free from graphene and carbon nanotubes.

23. The method of claim 16, wherein the detection sensitivity achieves a limit of detection (LOD) below 1 μg/L of manganese ions.

24. A method of fabricating an electrochemical sensor for manganese ion detection, comprising electrochemically depositing a composite consisting essentially of molybdenum disulfide (MoS₂) and chitosan onto a screen-printed carbon electrode (SPCE).

25. The method of claim 24, wherein MoS₂ and chitosan are mixed in equal volumes before deposition.

26. The method of claim 24, wherein the deposition is performed at a potential of +0.7 to +1.1 V for 30 to 900 seconds.

27. The method of claim 24, further comprising drying each deposited layer for approximately 4 minutes with heat.

28. The method of claim 24, wherein the MoS₂ in the composite is exfoliated nanosheets.

29. The method of claim 24, wherein the chitosan is prepared from chitin by partial deacetylation.

30. The method of claim 24, wherein the SPCE is disposable and fabricated from plastic substrates.
